(19) United States
(12) Patent Application Publication
(10) Pub. No.: US 2023/0032069 A1
(43) Pub. Date: Feb. 2, 2023

(54) MECHANICAL PROSTHESIS WITH RECIPIENT PHYSIOLOGICAL AND PROSTHESIS STATUS ACQUISITION CAPABILITIES

(71) Applicants: John Michael HEASMAN, East Melbourne (AU); Joris WALRAEVENS, Mechelen (BE)

(72) Inventors: John Michael HEASMAN, East Melbourne (AU); Joris WALRAEVENS, Mechelen (BE)

(21) Appl. No.: 17/391,697

(22) Filed: Aug. 2, 2021

(51) Int. Cl. H04R 25/00; A61B 5/12; A61B 5/00; A61B 5/388

(52) U.S. Cl. CPC H04R 25/606; A61B 5/125; A61B 5/4836; A61B 5/388; A61B 5/686

(57) ABSTRACT

A hearing prosthesis system, including a direct acoustic cochlear stimulator (DACS) sub-system, and an electrophysiology measurement sub-system, wherein at least a portion of the DACS sub-system and at least a portion of the electrophysiology measurement sub-system are configured to be permanently implanted in a recipient underneath skin of the recipient.

### CROSS REFERENCE TO RELATED APPLICATIONS

The present application is a Divisional application of U.S. patent application Ser. No. 15/164,494, filed May 25, 2016, naming John Michael HEASMAN as an inventor, the entire contents of that application being hereby incorporated by reference herein in its entirety.

### BACKGROUND

Hearing loss is generally of two types, conductive and sensorineural. Sensorineural hearing loss is due to the absence or destruction of the cochlear hair cells which transduce sound into nerve impulses. Various hearing prostheses have been developed to provide individuals suffering from sensorineural hearing loss with the ability to perceive sound. For example, cochlear implants have an electrode assembly which is implanted in the cochlea. In operation, electrical stimuli are delivered to the auditory nerve via the electrode assembly, thereby bypassing the inoperative hair cells to cause a hearing percept.

Conductive hearing loss occurs when the natural mechanical pathways that provide sound in the form of mechanical energy to cochlea are impeded, for example, by damage to the ossicular chain or ear canal. For a variety of reasons, such individuals are typically not candidates for a cochlear implant. Rather, individuals suffering from conductive hearing loss typically receive an acoustic hearing aid. Hearing aids rely on principles of air conduction to transmit acoustic signals to the cochlea. In particular, hearing aids amplify received sound and transmit the amplified sound into the ear canal. This amplified sound reaches the cochlea in the form of mechanical energy, causing motion of the perilymph and stimulation of the auditory nerve.

Unfortunately, not all individuals suffering from conductive hearing loss are able to derive suitable benefit from hearing aids. For example, some individuals are prone to chronic inflammation or infection of the ear canal. Other individuals have malformed or absent outer ear and/or ear canals resulting from a birth defect, or as a result of medical conditions such as Treacher Collins syndrome or Microtia.

For these and other individuals, another type of hearing prosthesis has been developed in recent years. This hearing prosthesis, commonly referred to as a middle ear implant, converts received sound into a mechanical force that is applied to the ossicular chain or directly to the cochlea via an actuator implanted in or adjacent to the middle ear cavity.

### SUMMARY

In accordance with an exemplary embodiment, there is a method, comprising mechanically simulating a component of a recipient's ear system, and recording data based at least in part on electrophysiological signals associated with a function of a cochlea related to the stimulation, wherein the action of stimulating and the action of recording the signals is executed entirely using a device permanently implanted in a recipient.

In accordance with another exemplary embodiment, there is a device, comprising a hearing prosthesis including an external component and an implantable component, wherein the implantable component is configured to mechanically stimulate tissue of the recipient to evoke a hearing percept; at least one of the external component, the implantable component or the external component and the implantable component in combination is configured to at least one of: record data based on electrophysiological signals of the recipient resulting from mechanical stimulation of the component(s) by the implantable hearing prosthesis; or evaluate the data.

In accordance with another exemplary embodiment, there is a hearing prosthesis system, comprising: a direct acoustic cochlear stimulator (DACS) sub-system; and an electrophysiology measurement sub-system, wherein at least a portion of the DACS sub-system and at least a portion of the electrophysiology measurement sub-system are configured to be permanently implanted in a recipient.

In accordance with an exemplary embodiment, there is a method comprising post-operatively supplying test signals to an implanted hearing instrument located within a recipient; and post-operatively obtaining an electrical potential associated with at least one of a cochlea or an auditory nerve or a brain stem or a brain of the recipient in timed relation to respective supplied test signals, wherein the action of obtaining the electrical potential is executed using a component permanently implanted in the recipient.

### BRIEF DESCRIPTION OF THE DRAWINGS

Embodiments are described below with reference to the attached drawings, in which:

FIG. 1 is a perspective view of an ear system of a recipient;

FIG. 2A is a perspective view of an exemplary hearing prosthesis in which at least some of the teachings detailed herein are applicable;

FIG. 2B is a perspective view of another exemplary hearing prosthesis in which at least some of the teachings detailed herein are applicable;

FIG. 3 is a perspective view of an exemplary implantable component of an exemplary hearing prosthesis in which at least some teachings detailed herein are applicable;

FIG. 4 is a perspective view of a component of the implantable component of FIG. 3;

FIG. 5 is a perspective view of an exemplary external component of an exemplary hearing prosthesis in which at least some teachings detailed herein are applicable;

FIG. 6 is a functional block diagram of an exemplary implantable component according to an exemplary embodiment;

FIG. 7 is a functional block diagram of a component of the implantable component of FIG. 6;

FIGS. 8A-9D represent exemplary actuator-coupling arrangements along with exemplary measurement electrode arrangements according some exemplary embodiments;

FIG. 10 represents an exemplary measurement electrode placement arrangement according to an exemplary embodiment;

FIG. 11 is a functional block diagram of an exemplary external component and an exemplary implantable component according to an exemplary embodiment;

FIG. 12 is a functional block diagram according to an exemplary component of the exemplary external component of FIG. 11;

FIG. 13 is a functional block diagram of an exemplary external component and an exemplary implantable component according to an exemplary embodiment;

FIG. 14 is a functional block diagram according to an exemplary component of the exemplary external component of FIG. 11;

FIG. 15 is a functional block diagram of an exemplary portion of an implantable component according to an exemplary embodiment;

FIG. 16 is a functional block diagram of another exemplary portion of an implantable component according to an exemplary embodiment;

FIG. 17 is a perspective view of an exemplary consumer electronics product according to an exemplary embodiment usable with the hearing prosthesis is detailed herein in at least some instances;

FIG. 18 is a quasi-functional diagram of a mechanical cochlear implant according to an exemplary embodiment, where the mechanical cochlear implant supports the electrodes;

FIG. 19 presents an algorithm according to an exemplary method according to an exemplary embodiment;

FIG. 20 presents another algorithm according to another exemplary method according to an exemplary embodiment;

FIG. 21 presents another algorithm according to another exemplary method according to an exemplary embodiment;

FIG. 22 presents another algorithm according to another exemplary method according to an exemplary embodiment;

FIG. 23 presents another algorithm according to another exemplary method according to an exemplary embodiment;

FIG. 24 presents another algorithm according to another exemplary method according to an exemplary embodiment; and

FIG. 25 presents another algorithm according to another exemplary method according to an exemplary embodiment.

### DETAILED DESCRIPTION

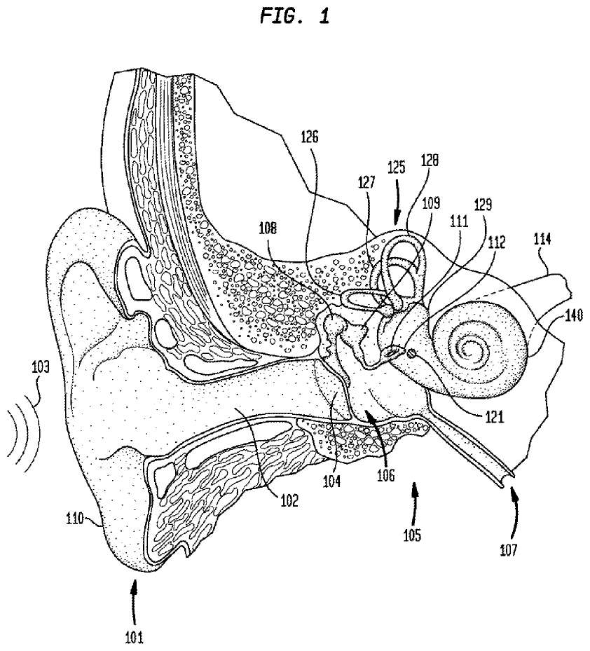

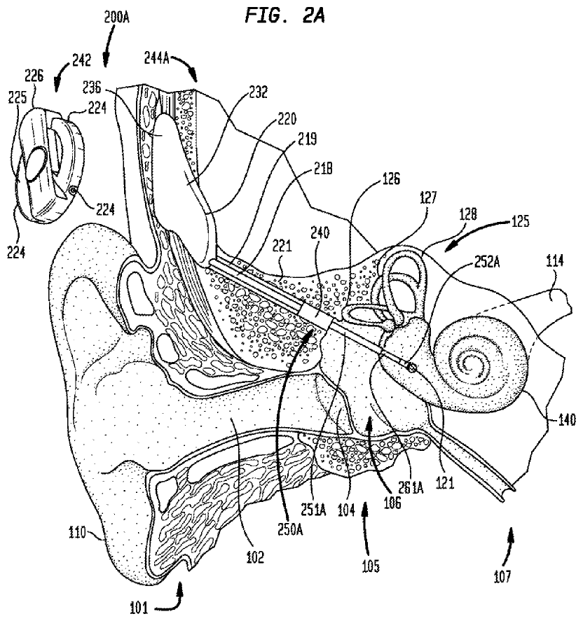

FIG. 1 is a perspective view of a human skull showing the anatomy of the human ear. As shown in FIG. 1, the human ear comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121. This vibration is coupled through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to the vibration of tympanic membrane 104. This vibration sets up waves of fluid motion of the perilymph within cochlea 140. Such fluid motion, in turn, activates hair cells (not shown) inside cochlea 140. Activation of the hair cells causes nerve impulses to be generated and transferred through the spiral ganglion cells (not shown) and auditory nerve 114 to the brain (also not shown) where they cause a hearing percept.

As shown in FIG. 1, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the orientation of the skull changes, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

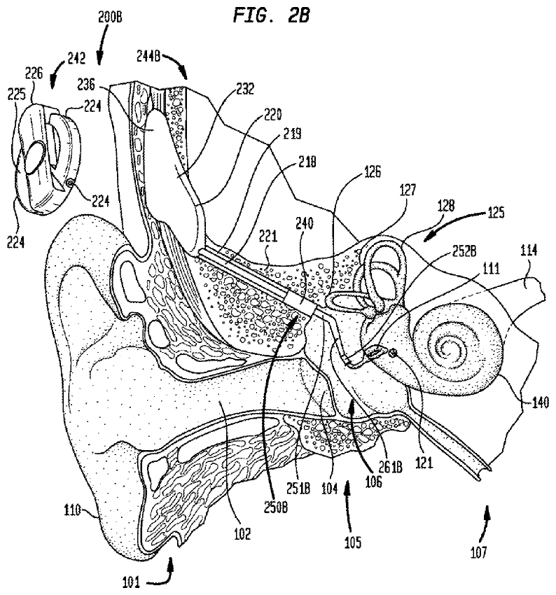

FIG. 2A is a perspective view of an exemplary direct acoustic cochlear stimulator 200A in accordance with an exemplary embodiment. Direct acoustic cochlear stimulator 200A comprises an external component 242 that is directly or indirectly attached to the body of the recipient, and an internal component 244A that is temporarily or permanently implanted in the recipient. External component 242 typically comprises two or more sound input elements, such as microphones 224, for detecting sound, a sound processing unit 226, a power source (not shown), and an external transmitter unit 225. External transmitter unit 225 comprises an external coil (not shown). Sound processing unit 226 processes the output of microphones 224 and generates encoded data signals which are provided to external transmitter unit 225. For ease of illustration, sound processing unit 226 is shown detached from the recipient.

Internal component 244A comprises an internal receiver/transmitter unit (hereinafter referred to as a communications unit) 232 including an inductance coil portion 236 (which is made, in some embodiments, out of silicone in which platinum coils (not shown) are embedded) and a stimulator unit 220, and a stimulation arrangement 250A in electrical communication with stimulator unit 220 via cable 218 extending through artificial passageway 219 in mastoid bone 221. Internal communications unit 232 and stimulator unit 220 are hermetically sealed within a biocompatible housing, and are sometimes collectively referred to as a stimulator/communications unit.

Internal communications unit 232 comprises an internal coil (not shown), and optionally, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated platinum or gold wire. The electrical insulation of the internal coil is provided by a flexible silicone molding (not shown). In use, implantable communications unit 232 is positioned in a recess of the temporal bone adjacent auricle 110.

In the illustrative embodiment of FIG. 2A, ossicles 106 have been explanted. However, it should be appreciated that stimulation arrangement 250A may be implanted without disturbing ossicles 106.

Stimulation arrangement 250A comprises an actuator 240, a middle ear prosthesis 252A and a coupling element 251A which includes an artificial incus 261B (represented in a quasi-functional manner—some additional details of these components are described below). Actuator 240 is coupled to mastoid bone 221 so as to be held in the interior of artificial passageway 219 formed in mastoid bone 221.

In this embodiment, stimulation arrangement 250A is implanted and/or configured such that a portion of middle ear prosthesis 252A abuts the round window 121. In an alternate embodiment, the middle ear prosthesis 252A can abut the oval window (not shown). In some alternate embodiments, stimulation arrangement 250B may alternatively be implanted such that the middle ear prosthesis 252A abuts an opening in horizontal semicircular canal 126, in posterior semicircular canal 127 or in superior semicircular canal 128. Any attachment regime that can enable a hearing percept to be evoked utilizing the stimulation arrangement 250A can be

As noted above, a sound signal is received by microphone(s) 224, processed by sound processing unit 226, and transmitted as encoded data signals to internal communications 232. Based on these received signals, stimulator unit 220 generates drive signals which cause actuation of actuator 240. The mechanical motion of actuator 240 is transferred to middle ear prosthesis 252A such that a wave of fluid motion is generated in the perilymph in the scala tympani of the cochlea. Such fluid motion, in turn, activates the hair cells of the organ of Corti. Activation of the hair cells causes appropriate nerve impulses to be generated and transferred through the spiral ganglion cells (not shown) and auditory nerve 114 to cause a hearing percept in the brain.

FIG. 2B depicts an exemplary embodiment of a middle ear implant 200B having a stimulation arrangement 250B comprising actuator 240 and a coupling element 251B. Coupling element 251B includes an ossicular replacement prosthesis or middle ear prosthesis 252B and an artificial incus 261B which couples the actuator to the middle ear prosthesis. In this embodiment, middle ear prosthesis 252B abuts stapes 111.

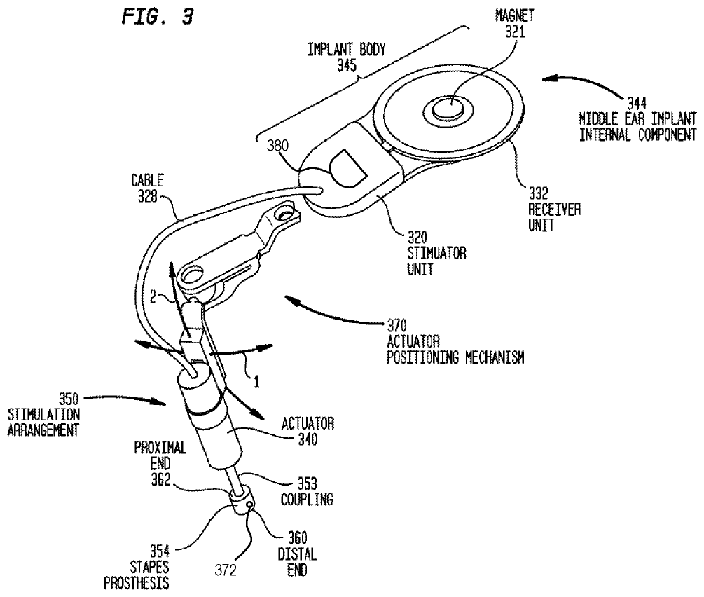

FIG. 3 is a perspective view of an exemplary internal component 344 of a middle ear implant in the form of a direct extra cochlear acoustic stimulator according to an exemplary embodiment. Internal component 344 comprises an internal communications unit 332, a stimulator unit 320, a stimulation arrangement 350, and an actuator positioning mechanism 370. As shown, communications unit 332 comprises an internal coil (not shown), and in some embodiments, a magnet 321 fixed relative to the internal coil. Internal communications unit 332 and stimulator unit 320 are typically hermetically sealed within a biocompatible housing. This housing has been omitted from FIG. 3 for ease of illustration, and hence the end of the actuator positioning mechanism 370, discussed in more detail below, which connects to the housing, is depicted with broken lines. This connection will be described with respect to later FIGS. Stimulator unit 320 also includes a return electrode 380 that is part of an implanted telemetry system, or, more generically, a status acquisition system, some features of which are described in greater detail below. Collectively, the internal communications unit 332, the stimulator unit 320 and the housing form an implant body 345.

Stimulator unit 320 is connected to stimulation arrangement 350 via a cable 328. Stimulation arrangement 350 comprises an actuator 340, a middle ear prosthesis 354 and a coupling element 353. A distal end of middle ear prosthesis 354 is configured to be positioned in one or more of the configurations noted above with respect to FIGS. 2A-2B. A proximal end of middle ear prosthesis 354 is connected to actuator 340 via coupling element 353 and the distal end of the prosthesis is directly or indirectly coupled to the cochlea. In operation, actuator 340 vibrates middle ear prosthesis 354. The vibration of middle ear prosthesis 354 generates waves of fluid motion of the perilymph, thereby activating the hair cells of the organ of Corti. Activation of the hair cells causes appropriate nerve impulses to be generated and transferred through the spiral ganglion cells and auditory nerve 114.

Middle ear implant internal component 344 further includes actuator positioning mechanism 370. As may be seen, actuator positioning mechanism 370 is connected to and extends from implantable body 345 and is configured to removably receive actuator 340. Stimulation arrangement 350 further includes a measurement electrode 372 that is part of the implanted telemetry system (which is a genus of a status acquisition system), some features of which are described in greater detail below.

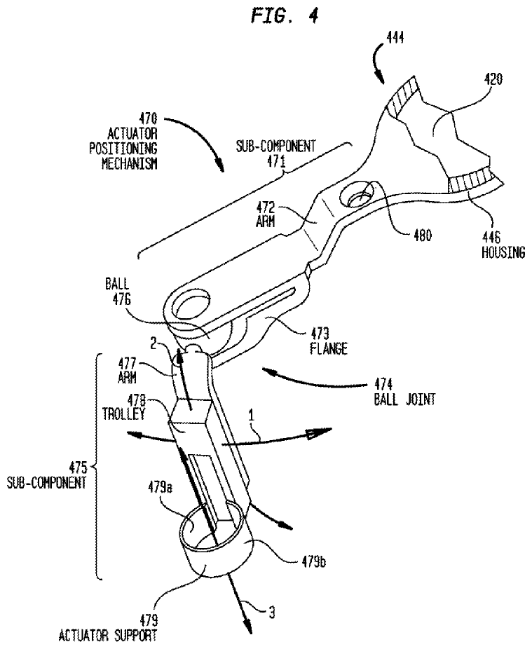

FIG. 4 depicts actuator positioning mechanism 470 as comprising two sub-components: extension arm 471 and extension arm 475. Sub-component 471 includes arm 472 which is an integral part of housing 446 (where the cross-hatching of housing 446 seen in FIG. 4 corresponds to the wall of the housing, as will be described in greater detail below). In an exemplary embodiment, arm 472 may be part of the same casting forming at least part of housing 446 (i.e., the arm 472 and at least a portion of the housing 446 form a monolithic component), although in an alternate exemplary embodiment, arm 472 may be a separate component that is attached to the housing 446 (e.g., via laser welding). In an exemplary embodiment, the casting may be made partially or totally out of titanium. In this regard, it is noted that the actuator support mechanism may be made partially or totally out of titanium, and the housing 446 may be made out of a different material. Sub-component 471 also includes flange 473 which forms a female portion of ball joint 474. In this regard, sub-component 475 includes the male portion of the ball joint 474, in the form of a ball 476, as may be seen. Ball joint 474 permits the ball 476 of sub-component 475 to move within the female portion, thereby permitting sub-component 475 to articulate relative to sub-component 471, and thus permitting actuator 340 to likewise articulate relative to middle ear implant internal component 344.

Ball joint 474 enables the actuator 340 to be positioned at an adjustably fixed location relative to the implantable body 345. In an exemplary embodiment, the ball joint 474 permits the location of the actuator 340 to be adjustable relative to the implant body in two degrees of freedom, represented by arrows 1 and 2 (first and second degrees of freedom, respectively), in FIGS. 3 and 4, although in some embodiments the joint may permit the location of the actuator 340 to be adjustable relative to the implant body in only one degree of freedom or in more than two degrees of freedom.

While actuator positioning mechanism 470 is depicted with a ball joint 474, other types of joints may be utilized. By way of example, the joint may comprise a malleable portion of a structural component of the actuator positioning mechanism 470 that permits the actuator 340 to be positioned as just detailed or variations thereof. In an exemplary embodiment, the joint is an elastically deformable portion or plastically deformable portion or is a combination of elastically deformable and plastically deformable portions so as to enable the adjustment of the location of the received actuator relative to the implant body in the at least one degree of freedom.

As noted above, actuator positioning mechanism 470 further includes sub-component 475. Sub-component 475 comprises ball 476 of ball joint 474, arm 477, trolley 478, and actuator support 479. Actuator support 479 is depicted as being in the form of a collar, and receives and otherwise holds actuator 340 therein, and thus holds the actuator 340 to the actuator positioning mechanism 470.

The collar has an exterior surface 479A and an interior surface 479B, configured to receive actuator 340. The interior diameter of the collar, formed by interior surface 479B is approximately the same as the outer diameter of the cylindrical body of actuator 340. The outer diameter of the collar, formed by exterior surface 479A, is sized such that the collar will fit into the artificial passageway 219. The length of the collar is shorter than the cylindrical body of the actuator 340, but in other embodiments, it may be the same length or about the same length or longer.

As noted, actuator support 479 and actuator 340 are configured to enable the actuator 340 to be removably secured to the actuator support 479, and thus the actuator positioning mechanism 470. This removable securement may be, in some embodiments, sufficient to prevent actuator 340 from substantially moving from the retained location in the actuator support 479, and the actuator positioning mechanism 470 is configured to prevent the actuator support 479 from substantially moving within the artificial passageway 219 during operation of the actuator 340. For example, the removable securement may be achieved via an interlock between the actuator 340 and the collar that provides retention sufficient to withstand reaction forces resulting from operation of actuator 340.

In an exemplary embodiment, the interlock is provided by an interference fit between inner surface 479A of the collar of actuator support 479 and an outer surface of actuator 340. In an alternate embodiment, the interlock is implemented as threads of inner surface 479A that interface with corresponding threads on the outer surface of actuator 340. In another embodiment, O-rings or the like may be used to snugly wrap around actuator 340 and snugly fit inside the collar of actuator support 479. Grooves on the actuator 340 and/or on the collar may be included to receive the O-ring. In other embodiments, compression of the O-ring between the actuator 340 and the collar provides sufficient friction to retain the components in the actuator support 479. In another embodiment, actuator support 479 or actuator 340 includes a biased extension that is adjusted against the bias to insert the actuator into the support. The extension may engage a detent on the opposing surface to interlock the actuator and the support. Other embodiments include protrusions and corresponding channels on opposing surfaces of the actuator and support. An exemplary embodiment includes a spring-loaded detent that interfaces with a detent receiver of the opposing surface to hold the actuator in the support or that extends behind the actuator once the actuator has been positioned beyond the detent. An alternate embodiment may utilize O-rings to interlock the actuator in the support. Adhesive may be used to interlock the actuator in the support. Any device, system, or method that will interlock the actuator in the support that will permit embodiments detailed herein and/or variations thereof to be practiced may be utilized in some embodiments.

The trolley 478, which is rigidly connected to actuator support 479, is configured to move linearly in the direction of arrow 3 parallel to the longitudinal direction of extension of arm 477. In this exemplary embodiment, arm 477 includes tracks with which trolley 478 interfaces to retain trolley 478 to arm 477. These tracks also establish trolley 478 and arm 477 as a telescopic component configured to enable the adjustment of the location of actuator support 479, and thus actuator 340 when received therein, relative to the housing 446 (thus the implant body), in at least one degree of freedom (i.e., the degree of freedom represented by arrow 3). It is noted that other embodiments may permit adjustment in at least two or at least three degrees of freedom. Thus, when the trolley component is combined with the aforementioned joint 474, the actuator positioning system enables the location of the actuator 340 to be adjustable relative to the implant body in at least two or at least three degrees of freedom.

Movement of the trolley 478 along arm 477 may be accomplished via a jack screw mechanism where the jack screw is turned via a screw driver or a hex-head wrench. Movement of the trolley 478 may also or alternatively be achieved via application of a force thereto that overcomes friction between the trolley 478 and the arm 477. Any device, system, or method that permits trolley 478 to move relative to arm 477 may be used in some embodiments detailed herein and variations thereof.

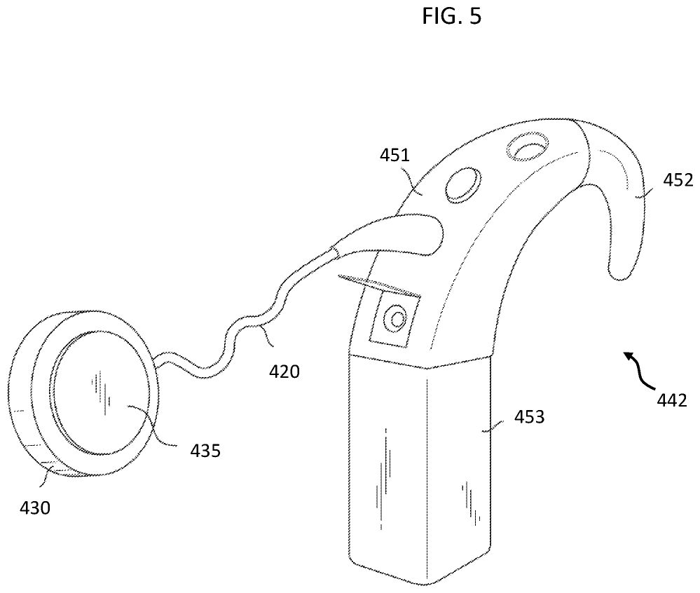

FIG. 5 depicts an alternate embodiment of an external component, external component 442, which corresponds to an external component usable as the external component FIGS. 2A-2B (i.e., in place of the button sound processor 242). In this embodiment, there is a behind-the-ear device that includes a behind the ear spine 451 having microphone ports, and ear hook 452, and a battery 453. In an exemplary embodiment, the microphone captures sound, and a sound processor in the behind-the-ear device converts that sound into an output signal which is fed to the headpiece 430 via cable 1420. That output signal energizes the inductance coil located in the headpiece 430 (which is held against the skin of the recipient via magnet 435) to evoke a hearing percept according to the teachings detailed herein.

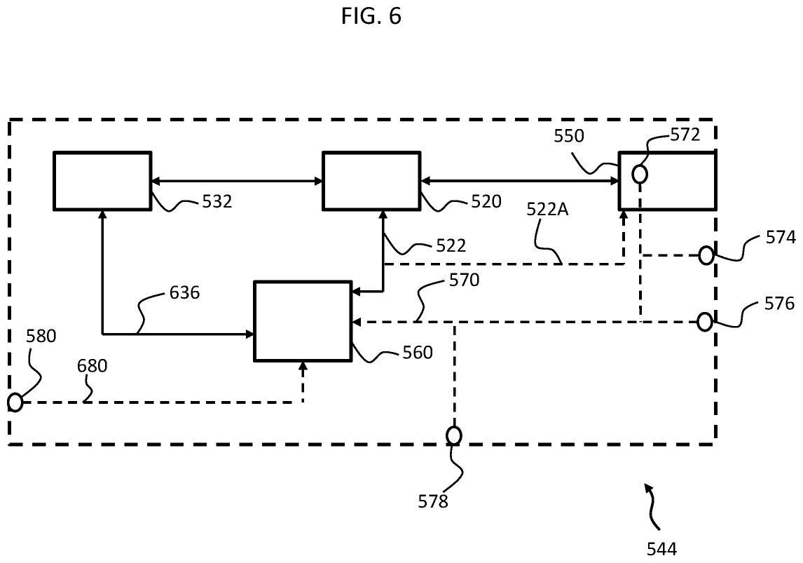

FIG. 6 depicts a functional block diagram of an internal component 544 usable as an internal component corresponding to those detailed herein and/or variations thereof. Internal component 544 includes a communications unit 532 corresponding to the communications unit 332 detailed above that receives and transmits respective RF inductance signals to communicate with the external component 242. In this regard, in an exemplary embodiment, receiver/transmitter unit 532 is configured to receive inductance signals from the external component 242 (or from another component configured to transmit such signals) so that the implantable component 544 can utilize those signals to evoke a hearing percept. It is further noted that in an alternate embodiment, the receiver/transmitter unit 532 is configured to receive inductance signals from the external component 242 (or from another component) so that the implantable component 544 can utilize those signals to implement the telemetric operations and methods detailed herein and variations thereof as will be described in greater detail below. The receiver/transmitter unit 532 is also configured to transmit inductance signals therefrom to the external component 242 (or to another component configured to receive such signals) so as to transmit the telemetric information gathered by the implantable component 544 as will be detailed below. As can be seen from FIG. 6, communications unit 532 is in two-way communication with stimulator unit 520, which corresponds to stimulator unit 320 above or the other stimulator unit detailed herein or any other type of stimulator unit that can have utilitarian value with back to the teachings detailed herein. Stimulator unit 520 is in turn in communication with stimulation arrangement 550, which corresponds to a stimulation arrangement 250 detailed above. FIG. 6 depicts two-way communication between units 520 and 550. That said, in an alternate embodiment, there is only one way communication between these two components. (It is noted at this time that any disclosure herein of one way communication corresponds to an alternate disclosure of two-way communication, and vice versa providing that the art enable such, unless otherwise specifically noted.) The communication between unit 520 and unit 550 permits the stimulation arrangement 550 to output mechanical forces to evoke a hearing percept based on received signals by the communications unit 532 to evoke a hearing percept according to the teachings detailed above.

Still with reference to FIG. 6, implantable component 544 includes a test unit 560 that can correspond to a processor or the like configured to implement testing according to the teachings detailed herein and/or variations thereof. It is noted that while the test unit 560 is depicted as being separate from the stimulator unit 320, in some alternate embodiments, the units are integral with one another. Any arrangement that can enable the teachings detailed herein and/or variations thereof to be practiced can be utilized in at least some exemplary embodiments. As can be seen, test unit 560 is in two-way communication with the communications unit 532 via signal line 636. Test unit 560 is also in two-way communication with stimulator unit 320 via signal line 522 and/or optionally with stimulatory arrangement 550 via signal line 522A (for economy of text, reference below will be made to signal line 522 placing units 520 and 560 in signal communication with one another, but any such disclosure also refers to a disclosure of utilizing alternatively and/or in addition to this signal line 522A to place unit 560 into communication with unit 550).

Test unit 560 is also in communication with one or more of measurement electrodes 572, 574, 576, and/or 578 (or more) via signal line 570, and is also in communication with one or more reference electrodes 580 corresponding to, in this exemplary embodiment, the reference electrode 580. It is noted that while the embodiment depicted in FIG. 3 presents the reference electrode 580 as being part of the stimulator unit 320, or otherwise supported by the housing that houses the stimulator unit 320, in an alternate embodiment, such as depicted here in FIG. 6, the return electrode 580 is located away from the stimulator unit 320. Any placement of the return electrode 580 that can enable the teachings detailed herein and/or variations thereof can be utilized in at least some exemplary embodiments.

It is briefly noted that some embodiments according to the teachings detailed herein are practiced without any electrodes located in the cochlea. That is, all electrodes are located outside the cochlea and are thus extra cochlear electrodes. Thus, an exemplary embodiment entails executing some or all of the teachings detailed herein and/or variations thereof in a noninvasive manner with respect to the cochlea (although embodiments will include an invasive process or device associated with the recipient in general). In an exemplary embodiment, the electrodes are arrayed or otherwise positioned so as to measure or otherwise detect signals indicative of the cochlear function.

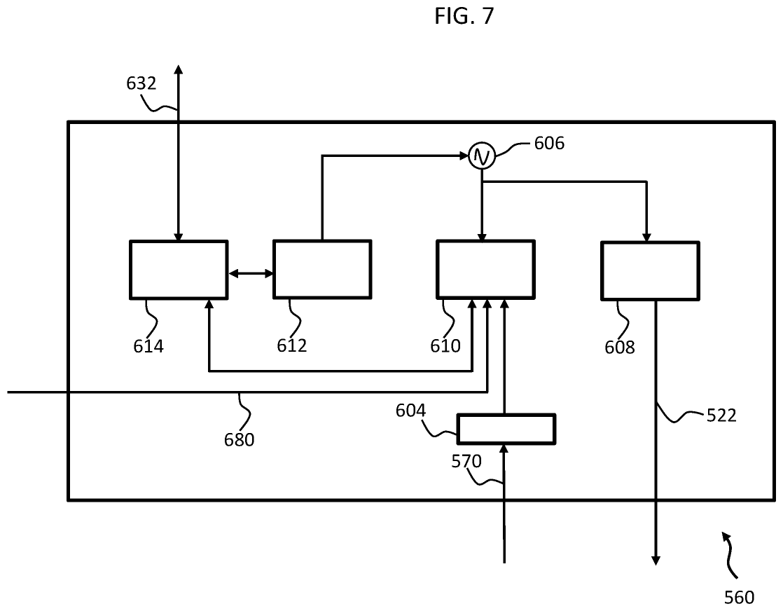

FIG. 7 presents additional details of test unit 560. It is again noted that the teachings associated with the test unit 560 are exemplary in nature, and can be implemented in any manner that will enable the teachings detailed herein. Indeed, the teachings detailed herein are presented for purposes of compactness and ease of understanding by referring to a “test unit.” It is noted that one or more or all of the functionalities detailed herein associated with the test unit can be distributed through other portions of the prosthesis. In this regard, any prosthesis that enables the functionality detailed herein can be utilized in at least some exemplary embodiments. With this in mind, an exemplary embodiment includes a measurement system having test unit 560 that includes an electrophysiology measurement (EP) device, 604, such as an electrocochleography (EC) measurement device, interconnected thereto. The electrophysiology measurement device 604 is configured to measure the electrical potential(s) associated with the cochlea and/or auditory nerve in response to test signals that are generated by the test unit 560 and supplied to the stimulation arrangement 550 (either directly via signal line 522A or indirectly via signal line 522, where stimulator unit 320 receives the output from the test unit 560 and generates stimulation signals based on that output that are utilized to actuate the stimulator arrangement 550). It is noted that while the embodiments detailed herein concentrate on the electrical potential, alternate embodiments can be utilized to measure other features associated with the cochlea and or auditory nerve. Any measurement of any physiological feature that can enable the teachings detailed herein and are variations thereof to be practiced can utilize in at least some exemplary embodiments. Herein, any disclosure of the measurement of electrical potentials with the sensation of electrical potentials corresponds to a disclosure of the measurement and/or sensation of any electrical phenomenon associated with electrophysiology (e.g., electrocochleography) and/or the disclosure of a measurement and/or sensation of any electrical phenomenon associated with electrophysiological signals that can enable the teachings detailed herein and/or variations thereof (e.g., measurement of higher evoked potentials (e.g., higher than ECOG)).

In an exemplary embodiment, the measured electrical potential(s) may be output as measurement signals by the electrophysiology measurement device 604 to the test unit 560 and processed/output to a user to assess whether a desired positional interface between the stimulation arrangement 350 and a middle ear component of a patient (e.g. a member of the ossicular chain) or inner ear of a patient is present. For example, in middle ear applications, such output is utilized in an exemplary embodiment by a healthcare professional during implantation procedures as a basis to determine whether to advance or retract the actuator of the stimulation arrangement 350 relative to a middle ear component. That said, as will be described in greater detail below, in other exemplary embodiments, such output is utilized by medical personnel in a post operative manner as a basis to determine whether to adjust a location of the actuator of the stimulation arrangement 350 (or to adjust another feature of the implantable component and/or the prostheses in general). Indeed, as will be described in greater detail below, in some exemplary embodiments, the output is utilized as a basis for fitting the prosthesis to the recipient. For example, in an exemplary embodiment, the gain of the prosthesis, or, more accurately, the gain regime (e.g., the algorithm used by the processor to set gain for given frequencies, which gain can be different for different frequencies), is adjusted based on the output. Indeed, in an exemplary embodiment, gain changes will be made in the post-operative adjustments to the prosthesis that are linked to automated implementation of at least some of the teachings detailed herein (and non-automated implementations as well). Moreover, as will be described in greater detail below, in other exemplary embodiments, such output are utilized by a recipient of the device himself or herself as a basis to make adjustments, or at least as a basis to notify the recipient that adjustments are utilitarian (e.g., thus prompting the recipient to obtain the services of a healthcare professional so as to make such adjustments (or even to remove the implantable component entirely in the case of a “catastrophic” scenario), as will be described in greater detail below. Note also that in some alternative exemplary embodiments (which can be combined with the embodiments just detailed, the outputs are utilized to monitor the everyday (or everyweek or everymonth, etc.) functionality of the prosthesis and/or to monitor the everyday (or everyweek, etc.) physiological features of the recipient.

More particularly, the electrophysiology measurement device 604 may be provided to measure or otherwise detect/sense cochlear microphonic, summating potential and/or compound action potential of the auditory nerve and the auditory nerve neurophonic in response to the noted test signals. The cochlear microphonic, summating potential is the electrical potential generated at the hair cell level in the cochlea. In some exemplary embodiments, such summating potential has a predeterminable latency range following stimulation. Further, the summating potential can have, in some exemplary embodiments, a predeterminable durational range (e.g., directly related to the test signal duration) and predeterminable absolute amplitude range. Such predeterminable ranges are employed by test unit 560 in at least some exemplary embodiments to facilitate processing of the measured potential values output by electrophysiology measurement device 604.

The action potential of the auditory nerve is an electrical response that is generated by the cochlear end of the VIII cranial nerve and is typically viewed as representing the summed response of the synchronous firing of thousands of auditory nerve fibers. That is, the size of the action potential reflects the number of nerve fibers which are firing simultaneously. In the absence of adverse pathology, the action potential can have a predeterminable latency range (e.g., about 1.30 milliseconds to 1.70 milliseconds). Its duration can also have a predeterminable range (e.g., about 0.80 milliseconds to 1.25 milliseconds), with a predeterminable absolute amplitude range (e.g., between about 0.60 millivolts and 3.00 millivolts). Such predeterminable ranges can be employed in some exemplary embodiments by test unit 560 to facilitate processing of the measured potential output from electrocochleography measurement device 604. Is noted that measurement signal values corresponding with the measured magnitude of the summating potential and/or action potential and/or a ratio thereof can be extracted and processed by the test unit 560 in at least some exemplary embodiments to assess the interface between the implantable transducer and middle ear component or inner ear of the recipient, e.g. the actuator of stimulation arrangement 550. As will be described in greater detail below, this assessment of the interface is utilized in some exemplary methods to determine whether or not adjustments to the prosthesis can have utilitarian value relative to not making adjustments to the prosthesis, etc.

In some embodiments, to enable measurement of the summating potential and/or action potential, the test unit 560 comprises one or more measurement electrodes 572, 574, 576, and/or 578. As shown in FIG. 5 and other FIGs. later introduced, an electrocochleography measurement electrode 572, 574, 576, and/or 578 can be positioned at a variety of locations (as is also the case with other types of electrography measurement electrodes).

Referring again to FIG. 6, the test unit 560 can comprise a signal generator 606, a reference transmitter 608, a signal processing unit 610, a test control processor 612, and a communication unit interface 614 that communicates with the communication unit 532 via signal line 632. By way of example, the test control processor 612 may provide signals for setting signal generator 606 to output reference signals at a predetermined frequency, or plurality of frequencies across a predetermined range, or a broadband reference signal, e.g., a click. The output reference signals may be provided to the reference transmitter 608, which in turn outputs test signals to the particular stimulation arrangement 550 (either directly or via the stimulator unit 320—to be clear, irrespective of how the output of the reference transmitter is provided, the end result is the driving of the implantable transducer, e.g., the stimulation arrangement 550 in the middle ear) and the signal processing unit 610. The signal processing unit 610 can analyze and/or store data based on the signals so as to enable an evaluation of the performance and positioning of the hearing prosthesis and/or a physiological aspect of the recipient. In an exemplary embodiment, the unit 610 is in communication with a non-transitory computer-readable media having recorded thereon, a computer program for executing one or more or any of the method actions detailed herein associated with the unit 610. In certain applications, it is utilitarian for the test control processor 612 to provide signals to the signal generator 606 to output reference signals that are swept across or inherently broadband to encompass a predetermined frequency range (e.g., a frequency range that encompasses a predetermined or determinable resonant frequency of an implantable stimulation arrangement 550). Any arrangement of test signals and control regimes that will enable the teachings detailed herein and/or variations thereof to be practiced can be utilized in at least some exemplary embodiments.

It is briefly noted that while the embodiment of FIG. 6 is presented in terms of the test unit 560 being a part of the same internal component as the stimulator unit (i.e., both are housed in the same housing), in some other embodiments, test unit 560 is located in a separate housing that is separate from the internal component that houses the stimulator unit, the test unit 560 being in signal communication with the communication unit and/or the stimulator unit via an electrical feedthrough. That said, in other embodiments, the test unit 560 can include its own communication unit so as to enable communication with the external component irrespective of the communication unit used by the stimulator unit. Any arrangement that can enable the teachings detailed herein and/or variations thereof to be practiced can be utilized in at least some exemplary embodiments.

It is briefly noted that while the embodiments presented above have been described in terms of the utilization of the signal generator 606 generating a signal, in an alternative embodiment, a signal upon which the basis of the telemetric teachings detailed herein are implemented can be based on a sound that is captured by the sound capture device of the hearing prosthesis, whether that be located outside of the recipient in the case of a partially implantable hearing prosthesis or within the recipient in the case of a fully implantable hearing prosthesis.

As noted above, the electrophysiology measurement device 604 provides, in some exemplary embodiments, measured electrical potential values to test unit 560 and/or another device of the prosthesis. More particularly, the measured potential values are provided to the signal processing unit 610 in the exemplary embodiment presented in FIG. 7. In turn, the signal processing unit 610 can process the measured potential values in accordance with preset algorithms. For example, utilizing the stored reference signal information and stored algorithms corresponding with one or more of the above noted predeterminable ranges, the signal processing unit 610 is configured in some embodiments to selectively extract the summating potential and/or action potential from the measured potential values. Still further, the processing unit 610 is further configured to process the extracted values (e.g., average the values and/or otherwise successively compared these values to determine whether and/or when a predetermined threshold or maximum value is reached (e.g. thereby indicating a desired interface). Concomitantly, the values obtained via processing at signal processing unit 610 can be output via interface 614 to the communication unit 532 and thus to external component 242 or other such device so that a healthcare professional can utilize such output as will be described in greater detail below.

It is briefly noted that while the embodiments presented above are directed toward utilization of the implantable component 544 with the external component 242 which is in the form of a button sound processor but can also be in the form of a typical BTE device with a headpiece, in some alternative embodiments, the teachings detailed herein can be utilized during surgery with a device that includes an RF coil that communicates with the RF coil of the communication unit 332. For example, there is an exemplary embodiment that includes a method where, during an implantation procedure of the implantable component, a healthcare professional utilizes the output from interface 614 accessed via the utilization of an external RF coil to assess whether and when the actuator 340 has been located to achieve the desired interface within the middle ear of a patient. In another approach, values obtained via processing at signal processing unit 610 (or some other unit) are used in conjunction with preset algorithms to provide a control signal to a transducer positioning apparatus (e.g., one or more motors or piezoelectric positioners attached to actuator positioning mechanism 370—described in greater detail below) to realize at least partially automated positioning of the implantable transducer. Further, following implantation, the noted interface may be assessed from time-to-time by comparing currently measured values with previously measured values (e.g., the previously measured values having been obtained at the time of implantation) or any other comparison that can have utilitarian value with respect to the teachings detailed herein and/or variations thereof.

FIG. 6 depicts various conceptual locations of the measurement electrode(s) used in at least some exemplary embodiments. Electrode 572 is conceptually depicted as being located on the stimulation arrangement 550. Electrodes 574 and 576 are depicted, again conceptually, as having different distances from the stimulation arrangement 550, representing different placements of the electrodes relative to the stimulation arrangement 550. Electrode 578 is positioned furthest of all electrodes away from stimulation arrangement 550. Some exemplary embodiments of the placements of the measurement electrodes will now be briefly described.

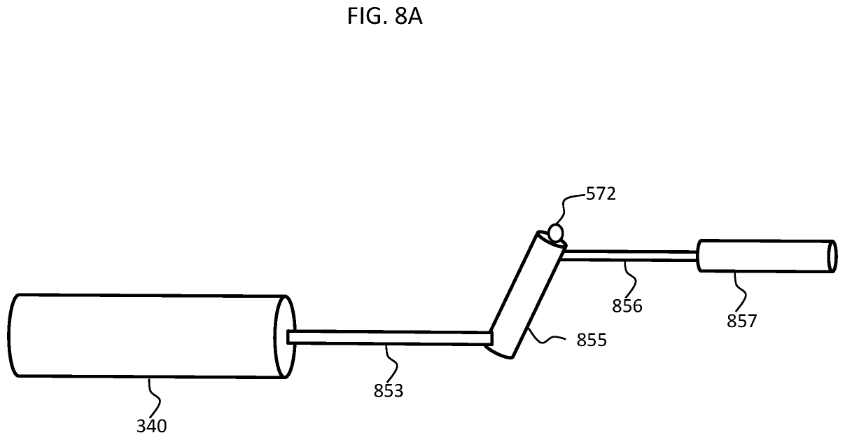

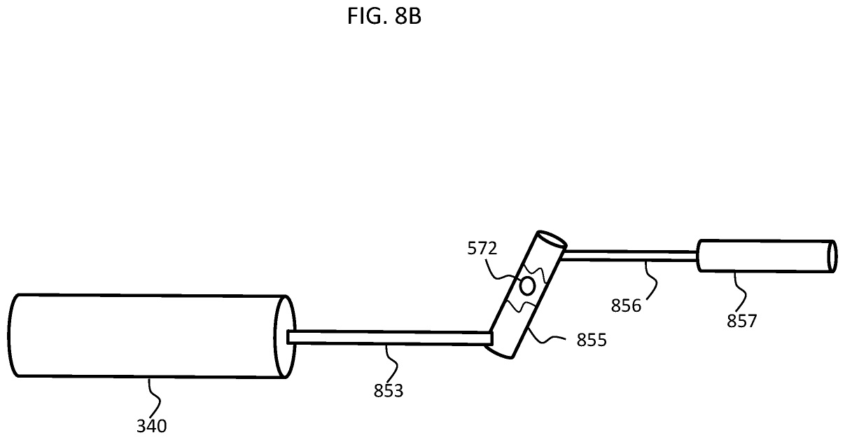

FIG. 8A depicts a portion of an exemplary stimulation arrangement including an actuator 340, corresponding to the actuator 340 of FIG. 3. FIG. 8A presents a coupling 853 extending from the actuator 340, which coupling is in the form of a rod that is coupled to the actuator 340. An artificial incus 855 is connected to the rod 853, and a second rod, rod 856, is also connected to the artificial incus 855. The middle ear prosthesis 857 is connected to the rod 856, as can be seen. In an exemplary embodiment, the middle ear prosthesis 857 is directly connected to the round window of the cochlea. In this regard, in an exemplary embodiment, the configuration of FIG. 8A corresponds to the arrangement of FIG. 2A detailed above. FIG. 8A depicts an exemplary location of the measurement electrode 572. As can be seen, electrode 572 is located on an end surface of the artificial incus 855, where the middle ear prosthesis 857, rod 856, and the pertinent portions of the artificial incus 855 are conductive, or at least there exists conductive components so as to establish a path between the tissue of the recipient and the electrode 572. Conversely, FIG. 8B depicts a conceptual version of an embodiment where the electrode 572 is integrated within the artificial incus 855, again where the middle ear prosthesis 857, rod 856, in the pertinent portions of the artificial incus 855 are conductive, or the pertinent conductive components are located so as to establish a conductive path from the tissue to the electrode. Note that while the embodiments of FIGS. 8A and 8B presents the electrode 572 as a distinct component from the artificial incus 855, in an alternate embodiment, the electrode and the artificial incus part are integrated together as a single component. That is, in an exemplary embodiment, the artificial incus is also a measurement electrode, or at least a portion of the artificial incus is such.

It is briefly noted that the embodiments of FIGS. 8A and 8B do not depict the electrode lead that extends from electrode 572 to test unit 560 for purposes of clarity. That said, in some alternate embodiments, the material and the design of the stimulation arrangement 550 is such that the electrode lead is integral to that arrangement.

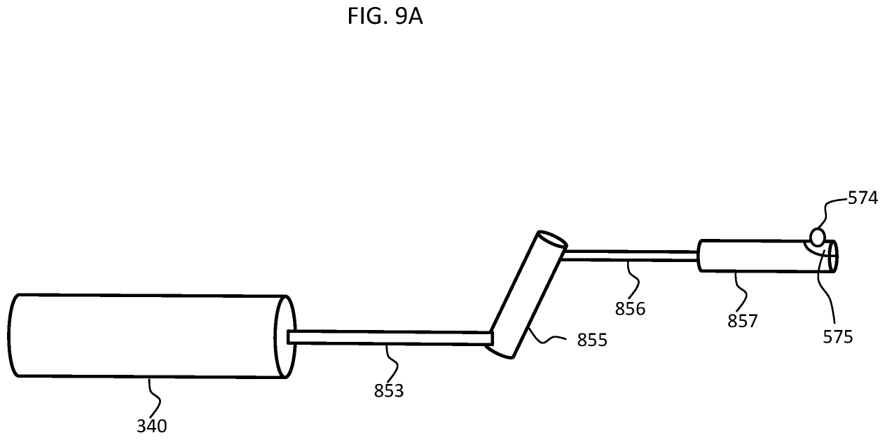

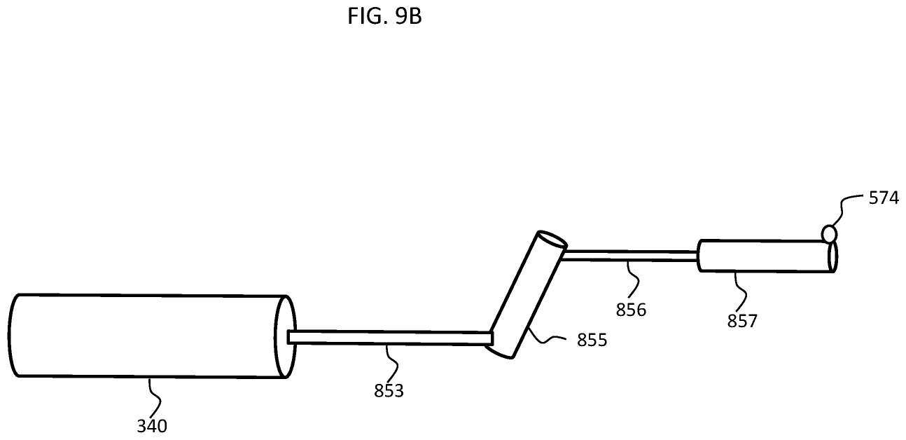

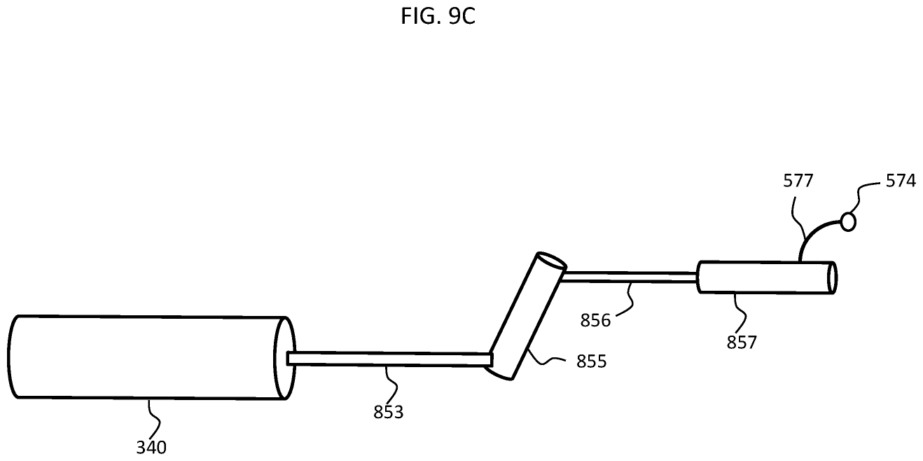

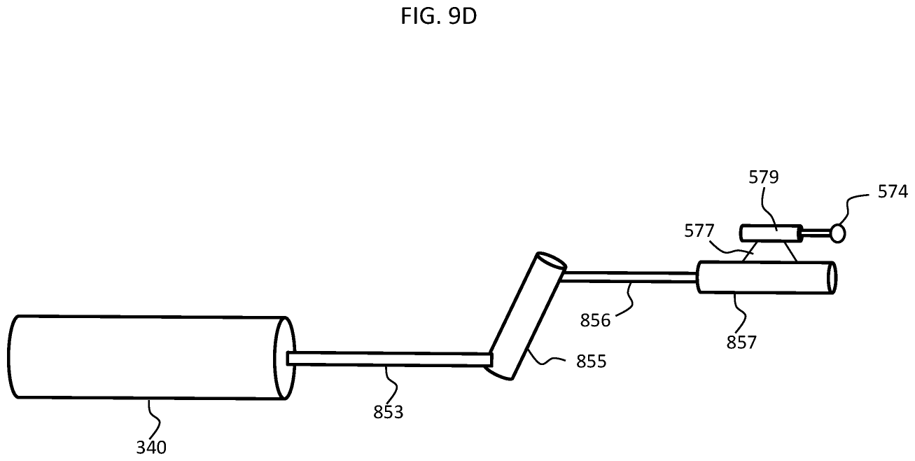

FIG. 9A depicts an alternate embodiment where the measurement electrode 574 is located on the middle ear prosthesis 857. When the middle ear prosthesis 857 is attached to the round window of the cochlea, the result is that the electrode 574 (which is represented by way of example only and not by way of limitation as a ball electrode) is placed near the round window of the cochlea. FIG. 9B depicts yet another alternate embodiment where the measurement electrode 574 is located at the very end of the middle ear prosthesis 857. In this embodiment, the configuration is such that the electrode 574 is in direct contact with the round window when the middle ear prosthesis 857 is attached thereto. This as opposed to the embodiment of FIG. 9A, where the electrode 574 does not directly contact the round window (in an exemplary embodiment, the middle ear prosthesis 857, or portion thereof, is conductive, as is represented by way of example only by cladding 575 in FIG. 9A which is cladded to the prosthesis 857 and establishes a conductive path from the round window to the electrode 574). FIG. 9C depicts an alternate embodiment where support structure 577 extends from the middle ear prosthesis 857 that supports a ball electrode 574 (or some other type of electrode) against an outside of the cochlea at a location near but not in direct contact to the round window. In this exemplary embodiment, structure 577 is a flexible structure that is configured to flex so as to accommodate the actuation of the middle ear prosthesis 857 relative to the fixed location of the outside of the cochlea to which ball electrode 574 is fixed (e.g., via surgical glue, stiches or by some other structure). In an alternate embodiment, structure 577 is a structure that is configured to press the electrode 574 against the outside of the cochlea so as to hold the electrode 574 against the tissue, which can have utilitarian value in embodiments where the ball electrode 574 is not fixed to the tissue (but can also have utilitarian value in embodiments where the ball electrode 574 is fixed to the tissue).

FIG. 9D depicts another exemplary embodiment where a support structure 577 supports a spring-loaded plunger 579 that supports the electrode 574 at the end of a spring-loaded cylinder. In this embodiment, plunger 579 is configured to accommodate the movements of the middle ear prosthesis 857 relative to the location where the ball electrode 574 is positioned against the outside of the cochlea. In this regard, the configuration of the plunger 579 is such that the force applied to the electrode 574 against the tissue of the recipient at a location proximate the round window is always greater than any force that would pull the electrode 574 away from the tissue resulting from the movement of the middle ear prosthesis 857 away from the cochlea during actuation thereof. In an exemplary embodiment, the entire stimulation arrangement is configured so as to account for any resistance or dampening effects, etc., of the plunger 579.

It is also noted that while the embodiments depicted in the figures represent an electrode that is generally at least quasi-permanently attached to the implant, an alternative embodiment, the electrode can be removable from the implant/repositionable relative to the implant in a global manner (as opposed to the more local manner depicted above). By way of example only and not by way of limitation, the electrode can be supported by a clip or a bracket with a bolt or screw arrangement or the like that can be clipped/bolted, respectively, onto various components of the implant at various locations (e.g., clipped around prosthesis 857, clipped or bolted to the artificial incus 855, etc.) Accordingly, in an exemplary embodiment, there is a prosthesis that enables the healthcare professional or the like to position the electrode at different given locations on the implant so as to provide versatility and placement to account for changing situations/different situations from one recipient to another recipient. In an exemplary embodiment, the implant is configured such that there can be pre-positioned/predetermined locations to receive the bolts and/or clips (e.g., a threaded hole with a bolt therein, an indentation that prevents the clip from sliding along the device/rotating relative to the device, etc.). In other alternative embodiments, the implant is configured such that the healthcare provider can clip or otherwise attached the electrode at various locations that are not pre-repaired.

As with the embodiments of FIGS. 8A and 8B, the various leads are not depicted in FIGS. 9A-9D. Also, as with the embodiments of FIGS. 8A and 8B, the electrodes can be integrated or integral with the middle ear prosthesis 857 with respect to the pertinent embodiments where such can be enabled.

While the embodiments of FIGS. 8A to 9D represent measurement electrodes that are supported or otherwise part of the stimulation arrangement 550, in some alternate embodiments, the measurement electrodes are not supported and/or are not part of the stimulation arrangement 550. FIG. 10 depicts an exemplary embodiment of a prosthesis 1000 including an implantable component 1044 including extra cochlear electrodes 574, 576, and 578, where the implantable component 1044 is implanted in a recipient. It is briefly noted that the embodiment depicted in FIG. 10 is depicted as including three separate extra cochlear electrodes. In some embodiments, only one or two of these electrodes are present. Still further, in some other embodiments, more than three electrodes are present (and thus there are embodiments where electrodes are located at locations differently than those depicted in FIG. 10). Still further, while the embodiment of FIG. 10 is depicted as having the stimulation arrangement connected to the stapes 111 (thus rendering the embodiment of FIG. 10 generally corresponding to the embodiment of FIG. 2B), some alternate embodiments include the electrode arrangement seen in FIG. 10 and/or the variations detailed herein except with respect to the embodiment of FIG. 2A, where the stimulation arrangement is directly connected to the round window via the middle ear prosthesis. Corollary to this is that in at least some embodiments, the electrode arrangement seen in FIG. 10 and the variations thereof detailed herein are also variously combined with the electrode arrangements of FIGS. 8A-9D (and it is noted that the embodiments of FIGS. 8A-9D can be variously combined with each other irrespective of the combination of the embodiment of FIG. 10). It is briefly noted that with respect to the embodiment of FIG. 10, the electrode 574 can include a clip or bracket or the like that can be clipped onto structure/bolted onto structure of the recipient so as to enable the electrode 574 to be positioned at a location that has utilitarian value.

More specifically, FIG. 10 depicts a ball electrode 574 located proximate the round window 121 (which can have utilitarian value with respect to electrophysiological signals corresponding to acoustically evoked cochlear and auditory nerve potentials), a ball electrode 576 positioned near the stapedius muscle (which can have utilitarian value with respect to measuring or otherwise sensing or detecting electrophysiological signals corresponding to acoustic reflex thresholds) and a ball electrode 578 positioned near the implanted package (proximate the transceiver stimulator (the combination of unit 232 and 220—it is noted that any disclosure herein of a receiver also corresponds to a disclosure of a transmitter providing that such is enabled by the art, and vice versa, unless otherwise noted)—an electrode placed near the implanted package can have utilitarian value with respect to detecting electrophysiological signals relating to the auditory brainstem response). As with the embodiments of FIGS. 8A-9D, the embodiment of FIG. 10 does not depict the respective leads connecting the respective electrodes to the test unit 560 located in the implanted package/that is part of the implanted package for purposes of clarity.

It is noted that the embodiments presented above uniformly depict all electrodes of the implantable component is part of the test unit as being extra cochlear electrodes. That is, the electrodes are all located outside of the cochlea. That said, in some alternative embodiments, the teachings detailed herein and/or variations thereof can be utilized with one or more electrodes located within the cochlea. Still further, while the embodiments have generally been directed towards utilization of so-called ball electrodes, other types of electrodes can be utilized. Any arrangement that can enable the teachings detailed herein and/or variations thereof to be practiced can be utilized in at least some exemplary embodiments.

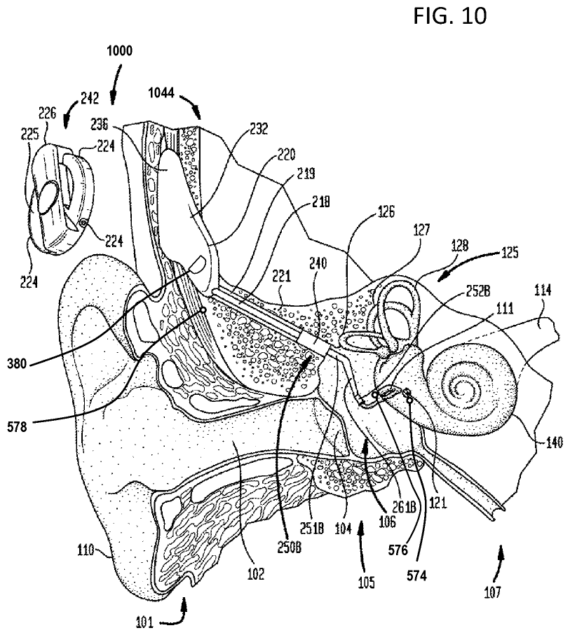

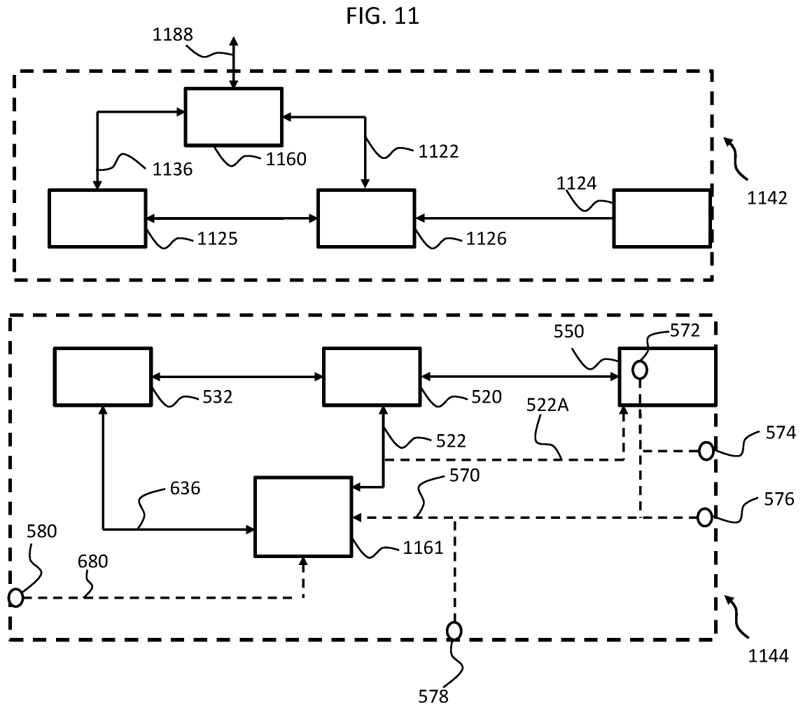

While the embodiments of FIGS. 6 and 7 have been presented in terms of a totally implantable test unit 560, FIG. 11 depicts an exemplary embodiment where the test sub-system of the hearing prosthesis is bifurcated between components that are implanted and components that are external the recipient but still part of the prosthesis (i.e., part of the external component). More specifically, FIG. 11 depicts a functional schematic of an external component 1142 according to an exemplary embodiment. In an exemplary embodiment, external component 1142 can correspond to the button sound processor of FIGS. 2A and 2B, or can correspond to the behind-the-ear device of FIG. 5, or can correspond to any other component having utilitarian value. External component 1142 includes a sound capture apparatus 1124, corresponding to any of the sound capture apparatuses as detailed above. Sound capture apparatus 1142, which can include multiple microphones, as detailed above, is in signal communication with the sound processor 1126. Sound processor 1126, which can correspond to any of the sound processors detailed above, processes the received signals into output signals which are provided to communications component 1125, which, in an exemplary embodiment, can correspond to a RF inductance coil and accompanying components as detailed above, by way of example. External component 1142 further includes test sub-system unit 1160, which is in signal communication with the communication component 1125 via signal line 1136 and is optionally in signal communication with the sound processor 1126 via signal line 1112. Some additional details of tests sub-system unit 1160 will be described below.

Implantable component 1144 is presented using like reference numbers with respect to components of the implantable component 544 detailed above with respect to FIG. 6. With respect to the schematic representing implantable component 1144, implantable component 1144 differs from the implantable component 544 in that instead of test unit 560, test subsystem unit 1161 is present in its place (for purposes of conceptual representation). Here, test sub-unit 1161 corresponds to measurement device 604 detailed above, and receives input from componentry located in the external component 1142, which input is provided via communication component 532 via signal line 636. That said, in some alternate embodiments, test sub unit 1161 includes further functionalities of the test unit 560 detailed above, such as by way of example only and not by way of limitation, the functionality of the signal processing unit 610, the functionality of the reference transmitter 608, the functionality of the test control processor 612, and/or the functionality of the signal generator 606. However in some other alternate embodiments, even the functionality of the measurement device 604 is bifurcated between the external component and the implantable component, as will be described further below.

In an exemplary embodiment, test sub-unit 1161 corresponds to a separate processor, while in other embodiments, test sub-unit corresponds to an electronics component configured to activate upon receipt of a signal via signal line 636 and/or via signal line 522. Any arrangement that can enable test sub-unit 1161 to operate according to the teachings detailed herein and/or variations thereof can be utilized in at least some exemplary embodiments.

Test sub-unit 1161 is in communication with one or more of measurement electrodes 572, 574, 576, and/or 578 (or more) via signal line 570, as is the case with respect to unit 560 of FIG. 6, and is also in communication with one or more reference electrodes 580 corresponding to, in this exemplary embodiment, the reference electrode 580.

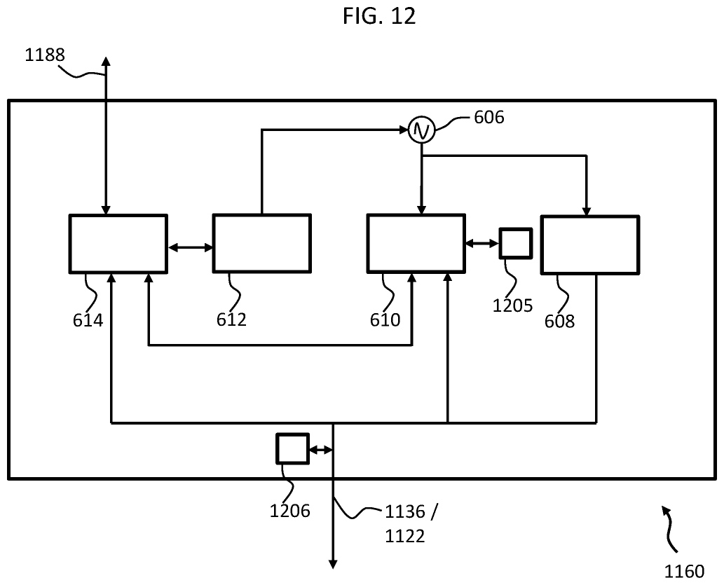

FIG. 12 presents additional details of test sub-unit 1160. It is again noted that the teachings associated with the test unit 1160 are exemplary in nature, and can be implemented in any manner that will enable the teachings detailed herein. It is noted that one, or more, or all of the functionalities detailed herein associated with the test sub-unit 1160 can be distributed through other portions of the prosthesis (such as being instead or in addition to this included in the test sub-unit 1161).

Test sub-unit 1160 is configured to interface with the measurement device 604 in a manner consistent with the embodiment of FIGS. 6 and 7 detailed above, except that communication between test sub-unit 1160 and the measurement device 604 of test sub-unit 1161 is established through the inductance link established between components 1125 and 532. It is further noted that in an exemplary embodiment, the features of the measurement device 604 are distributed between test sub-unit 1160 and test sub-unit 1161. For example, the analytical and/or adjustable features of the measurement device 604 can be located in the test sub-unit 1160, and the electrode signal reception features and/or signal boost features can be located in the test sub-unit 1161 (described further below).

Referring again to FIG. 12, the test sub-unit 1161 may comprise a signal generator 606, a reference transmitter 608, a signal processing unit 610, a test control processor 612, and a communication unit interface 614 that communicates with the communication unit 532 via signal line 1136, but can also communicate with the speech processor via signal line 1122.

It is briefly noted that while the embodiment of FIG. 11 is presented in terms of the test sub-unit 1161 being a part of the same internal component as the stimulator unit (i.e., both are housed in the same housing), in some other embodiments, test sub-unit 1161 is located in a separate housing that is separate from the internal component that houses the stimulator unit, the test unit 560 being in signal communication with the communication unit and/or the stimulator unit via an electrical feedthrough, although in other embodiments, the test sub-unit 1161 can include its own communication unit so as to enable communication with the external component irrespective of the communication unit used by the stimulator unit. Any arrangement that can enable the teachings detailed herein and/or variations thereof to be practiced can be utilized in at least some exemplary embodiments.

FIGS. 11 and 12 indicate that the test sub-unit 1160 is in two-way communication with the external environment of the external component 1142 via communication line 1188. In an exemplary embodiment, this can be established via a USB connection or the like. Any arrangement that can enable two-way communication with test sub-unit 1160 can be utilized in at least some exemplary embodiments. That said, in some alternate embodiments, there is only one way communication between test sub-unit 1160 and the external environment of the external component 1142. Note further that in some exemplary embodiments, while the communication with the external environment is presented as being directly with test sub-unit 1160, in an alternate embodiment, the communication is indirect (e.g., the communication can extend through sound processor 1126 etc.).

In an exemplary embodiment, communication line 1188 is utilized to obtain telemetric data and/or results of any analysis associated with the test units detailed herein. Additional details of this will be described below.

It is further noted that the arrangement of the external component 1142 of FIG. 11 can be applicable, in some modified versions, with the embodiments detailed above and/or without modification.

FIG. 12 indicates that the external component 1142 in general, and the test subunit 1160 in particular, includes a memory unit 1205 in signal communication with signal processing unit 610. In an exemplary embodiment, memory unit 1205 records events and/or data relating to the test unit(s) as will be described in greater detail below. In an exemplary embodiment, memory unit 1205 enables the memories stored therein to be accessed at a later date, either by the test system of the prosthesis and/or by a user, such as via communication line 1188. It is further noted that while memory unit 1205 is depicted as being in direct communication with processing unit 610, in an alternate embodiment, memory unit 1205 is in direct communication with other components of the test subsystem 1160 and/or other components of the prosthesis in general. It is further noted that additional memory units can be utilized in the subunit 1160, such as by way of example only and not by way of limitation, memory unit 1206, as can be seen. Also, it is noted that memory unit 1206 can be utilized instead of memory unit 1205, and that memory unit 1206 need not necessarily be part of the subunit 1160, but can be located external to the subunit 1160.

In an exemplary embodiment, the memory unit 1205 and/or the memory unit 1206 is a standard memory unit usable in consumer electronic products and/or other types of computers.

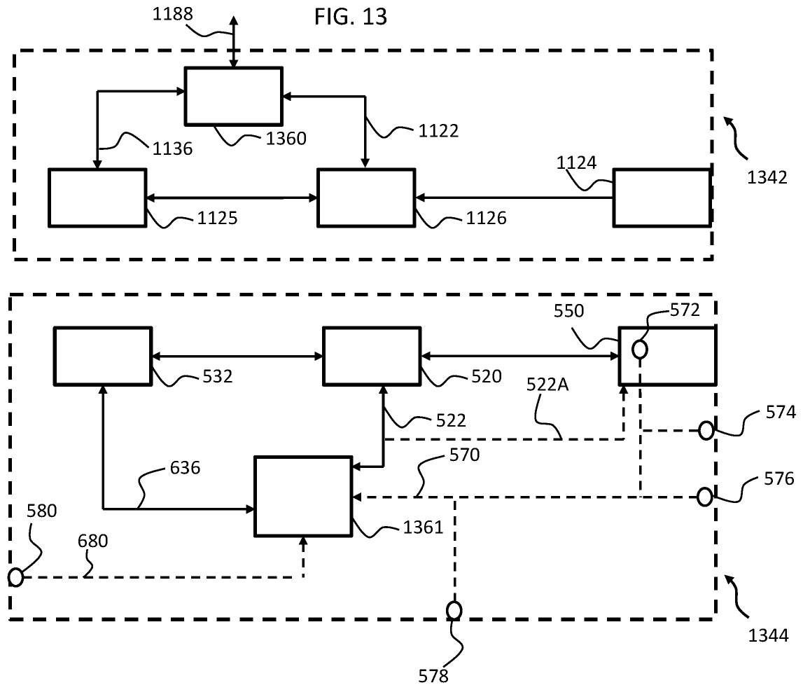

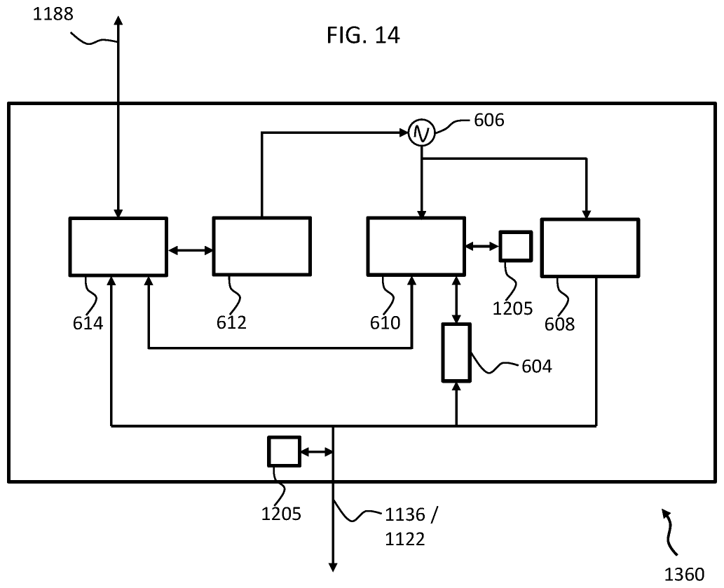

FIG. 13 depicts yet another exemplary embodiment of a prosthesis according to some exemplary embodiments. The embodiment of FIG. 13 corresponds to the embodiment of FIG. 11, with the exception that the test sub-unit 1360 of external component 1342 includes some additional functionalities than that of test sub-unit 1160 of the external component 1142, and with the exception that test sub-unit 1361 of implantable component 1344 includes fewer functionalities than that of test sub-unit 1161 of the external component 1144. More particularly, test sub-unit 1360 includes some of the functionality of the measurement device 604. Conversely, test sub-unit 1361 entails reduced functionality from that with respect to test sub-unit 1161. By way of example only and not by way of limitation, test sub-unit 1361 is a component that receives signals from the various measurement electrodes and compares the signals to the signal received from the reference electrode 580. In an exemplary embodiment, test sub-unit 1361 amplifies the resulting signal and outputs this signal to communications unit 532 so that it can be communicated to the external component 1342 in general, and ultimately to the measurement device 604 of external component 1360, as is functionally depicted in FIG. 14.

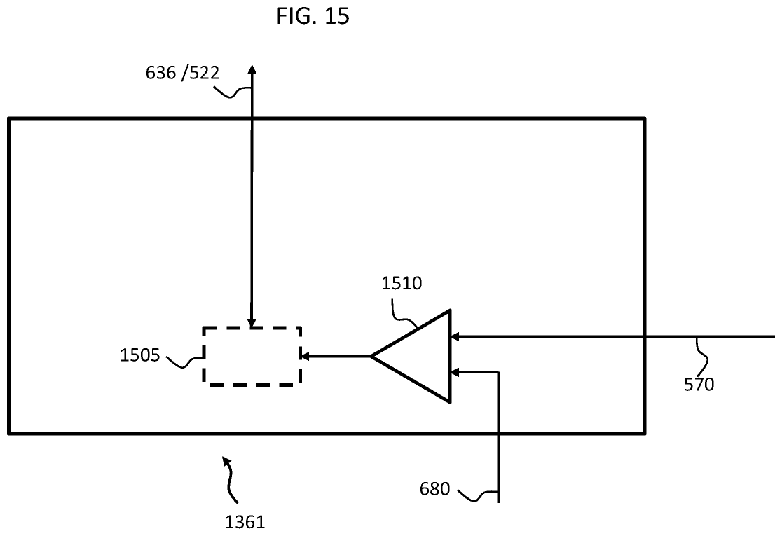

FIG. 15 depicts a quasi-functional arrangement of an exemplary embodiment of test subunit 1361. As is functionally depicted, input from the measurement electrodes is provided to sub-unit 1361 via signal line 570. This input is provided to an amplifier 1510. Along with input from the reference electrode 680. Amplifier 1510 outputs a signal that is indicative of the true difference between the two inputs. This output can be provided directly to the communication unit 536 and/or the processor 520. In an alternate embodiment, the output is not directly provided to these components, but instead is stored via storage unit 1505, which can entail a memory device such as any memory device that is usable to store electronic data. The storage in storage unit 1505 can be temporally catalogued and/or event catalogued. Upon a query from another component of the prosthesis and/or at a predetermined time and/or as a result of a predetermined event, the data in storage unit 1505 can be delivered to the communication unit and/or the processor for use and/or for transmission to the external component or to whatever component that is utilized to receive this data outside the recipient.

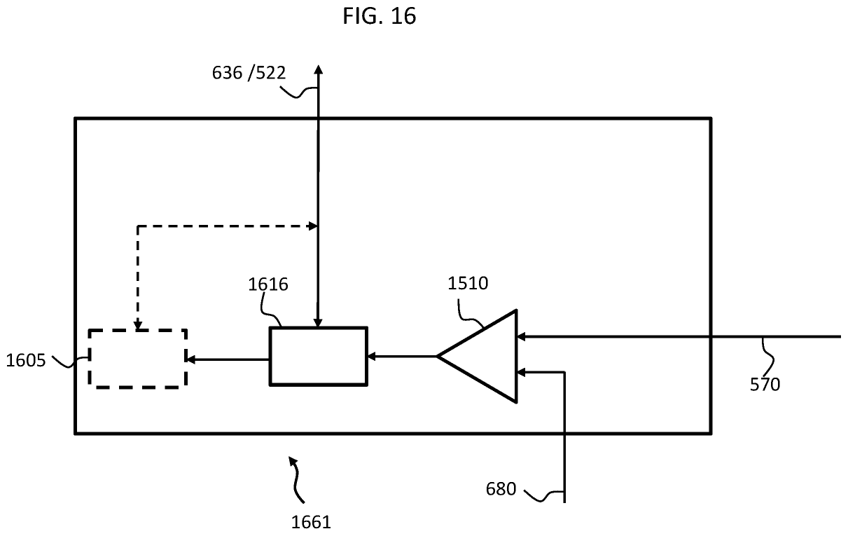

As noted above, the output of amplifier 1510 can be correlated to events. In this regard, FIG. 16 depicts an alternate embodiment of a test sub-unit 1661 that can replace test sub-unit 1361 described above. Here, test sub-unit 1661 includes a correlation block 1616, which receives input via signal lines 1636, and/or signal line 522 related to an event occurring in real time/contemporaneous with the input from the amplifier 1510. In an exemplary embodiment, the event input can correspond to data related to the signal provided by the reference transmitter 608 to the actuator (either directly or indirectly) of the prosthesis. Thus, in an exemplary embodiment, the output from amplifier 1510 can be correlated to the signals provided to the actuator that cause the actuator to stimulate tissue that resulted in the signal from the given measurement electrode. In an exemplary embodiment, the correlator block 1616 can then output correlated data back to the communication unit 536 and/or the processor 520. Alternatively, and/or in addition to this, this correlated data can be stored in memory unit 1605 and subsequently provided to the communication unit 536 and/or the processor 520 in the manner concomitant with that described above with respect to memory unit 1505.

It is further noted that the functionalities just described of the memory unit 1505 and/or 1605 can also be applicable to the memory units 1205 and 1206 described above. It is also noted that in an exemplary embodiment, the test sub-units of the external component include a correlator block having the functionalities described with respect to correlation block 1616.

In view of the above, in an exemplary embodiment, there is a device comprising a hearing prosthesis, including an external component (e.g., a behind the ear device such as 442, a button sound processor, etc., along with the functionality of, for example, 1142, 1342, etc.) and an implantable component (e.g., such as 344, 544, 1144, 1344, etc.). In an exemplary embodiment, the implantable component is configured to mechanically stimulate tissue of the recipient to evoke a hearing percept. In an exemplary embodiment, this can be achieved via a middle ear implant, and/or a mechanical cochlear implant, and/or a bone conduction device, although in some other embodiments, the implantable component is configured to directly stimulate an ear component of the recipient, such as the middle ear and/or the inner ear, and a bone conduction device indirectly stimulates such. In this exemplary embodiment, at least one of the external component, the implantable component, or the external component and the implantable component in combination is configured to at least one of record data based on electrophysiological signals of the recipient resulting from mechanical stimulation of the component(s) by the implantable hearing prosthesis or evaluate that data. With respect to the recordation of the data, such an exemplary embodiment can correspond to the embodiment of FIG. 13 with respect to the implantable component, and the embodiments of FIG. 12 with respect to the external component, and a combination of the two with respect to the embodiment of FIGS. 13-15 (where the test sub-unit 1361 includes memory 1505). With respect to the evaluation of the data, such an embodiment can correspond to the embodiment of FIG. 6 with respect to the implantable component (implantable component 544), and the embodiments of FIG. 11 with respect to the external component (external component 1142), and the combination of the implantable component and the external component with respect to embodiments utilize a combination of implantable component 544 and an external component 1142. Some additional details of the evaluation of the data will be described below.

It is briefly noted by the phrase “data based on electrophysiological signals,” this includes data directly corresponding to such signals (i.e., the raw data) and processed signals (as well as data corresponding to the result of subsequent processing of those processed signals). That is, if the underlying basis for the data is at least in part from the electrophysiological signals, this corresponds to data based on the signals.

In view of the above, some exemplary embodiments include an implantable component that is configured to sense electrical phenomenon of the recipient indicative of the electrophysiological signals of the recipient resulting from mechanical stimulation the implantable component is configured to sense electrical phenomena of the recipient indicative of the electrophysiological signals of the recipient resulting from mechanical stimulation. In an exemplary embodiment, this can be achieved via the various test units and/or test subunits detailed herein and variations thereof. In particular, in an exemplary embodiment of this exemplary embodiment, the implantable component includes one or more of the measurement electrodes along with one or more reference electrodes. As noted above, the electrodes can be part of the stimulatory assembly and/or other components of the implantable component. Any device, system, and/or method that will enable an implantable component to sense electrical phenomenon of the recipient indicative of the electrophysiological signals resulting from the mechanical stimulation can be utilized in at least some exemplary embodiments.

It is further noted that the ability to sense the electrical signals does not require any form of analysis or the like or even recordation of the sensation, that being covered by the ability to evaluate the data and the ability to record the data respectively. Accordingly, in an exemplary embodiment, the ability of the implantable component to sense electrical phenomena can be achieved via the embodiments of FIG. 15.

That said, as noted above, embodiments include a prosthesis that includes at least one of the external component, the implantable component, or these components in combination having the ability to evaluate the data based on the electrophysiological signals. It is noted that the aforementioned embodiment can include an embodiment where the external component and the implantable component can separately evaluate the data irrespective of the other, as well as an embodiment where the two components work in combination to evaluate the data. It is further noted that such an embodiment need not necessarily be able to record the data. That is, an embodiment can exist where the device analyzes the data and utilizes the data in real-time. That said, in some embodiments, this device has the ability to both record the data and evaluate the data.

In this regard, an exemplary embodiment includes a prosthesis that is configured to automatically adjust a feature of the implantable component based on the evaluated data. By way of example only and not by way of limitation, in an exemplary embodiment where the prosthesis utilizes the configuration of FIG. 3, the prosthesis can adjust the location of the stimulation arrangement 350 utilizing the arrangement of FIG. 4. In an exemplary embodiment, one or more miniaturized actuators can be included in the arrangement of FIG. 3 that are implantable with the recipient and can be utilized to move the stimulation arrangement 350 according to the various movement scenarios detailed above with respect to FIG. 4. By way of example only and not by way of limitation, based on the evaluation of the data based on the electrophysiological signals, the prosthesis can be configured to determine that the stimulation arrangement 350 is underloaded or overloaded, and adjust the position of the stimulation arrangement 350 automatically by controlling the various actuators of the implantable component to move the stimulation arrangement 350. That said, it is noted that in some alternate embodiments, the prosthesis is not configured to make a true determination that the stimulation arrangement 350 is under loaded or overloaded. In an exemplary embodiment, the prosthesis can be configured to evaluate the data and make adjustments based on, for example, a lookup table. That is, it is not necessary for the system to make an affirmative determination of the status or otherwise make an affirmative determination of the scenario afflicting the stimulation arrangement 350 in general, and its relationship with the tissue of the recipient in particular. In some embodiments, the prosthesis makes these adjustments according to an algorithm stored in the prosthesis. By way of example only and not by way of limitation, if the data corresponds to X, and the evaluation of the data indicates that X means that the stimulation arrangement 350 should be moved by Y, that corresponds to the evaluation of the data even though no determination is made of whether X means underloading or overloading.

Embodiments are not limited to simply addressing on the loading and/or overloading. Embodiments can also be directed towards addressing the angular alignment of the stimulation arrangement 350 with the tissue as well. Any repositioning or position adjustment of the stimulation arrangement 350 (or other arrangement, stimulation arrangement 350 being used by way of example only and not by way limitation for the purposes of illustrating this embodiment) can be included in the action of automatically adjusting the feature of the implantable component.

Note further that embodiments are not simply limited to the repositioning of the stimulation arrangement 350. Adjustment of a feature of the implantable component can further include adjustment of a mechanical feature of the stimulation arrangement 350. By way of example only and not by way of limitation, an adjustment can be made that adjusts the resonant frequency of the stimulation arrangement 350. Still further by example, an adjustment can be made that limits a frequency and/or an amplitude output of the stimulation arrangement 350. Moreover, it is noted that the ability of the prosthesis to adjust the implantable component is not limited to adjustments relating to the stimulation arrangement 350. In this regard, according to an exemplary embodiment, adjustments can also be made to the operation of the transceiver stimulator and/or the operation of the sound processor (in the case of a totally implantable system where the sound processor is included in the implanted receiver stimulator). By way of example only and not by way of limitation, adjustments can be made to reduce the amplitude of the output of the transceiver stimulator to the stimulation arrangement 350 for certain frequencies of, or for all frequencies, and/or to increase the output thereof for certain frequencies, or for all frequencies relative to that which might otherwise be the case.

It is further noted that, as detailed above, in an exemplary embodiment, the gain/gain regime of the prosthesis will be adjusted based on the data. It is briefly noted that any disclosure herein of adjusting a mechanical component/location of the actuator for a given scenario also corresponds to a similar disclosure correspond to the adjustment of a gain/gain regime of the prosthesis, where the specific text relating to the disclosure is not repeated for purposes of linguistic efficiency.

Note also that while the embodiments detailed herein have been up until now directed towards automatically adjusting a feature of the implantable component, in an alternate embodiment, this can also include automatic adjustment of a component of the external component (e.g., such as where the sound processor is in the external component as is the case in a partially implantable hearing prosthesis).

In another exemplary embodiment, a map setting of the prosthesis can be adjusted based on the evaluated data, etc. That is, different map settings can be implemented and/or a given map setting can be adjusted based on the evaluated data. For example, an evaluation of the data that is indicative of a physiological change in the recipient relative to that which was previously the case can warrant the utilization of a different map of the hearing prosthesis and/or can warrant an adjustment to a given map that is currently being utilized or otherwise was previously utilized prior to this evaluation. In an exemplary embodiment, this can be executed automatically. In this regard, in an exemplary scenario, weeks and/or months and/or years after the surgical implantation of the hearing prosthesis, an evaluation based on the data can indicate that a physiological change in the recipient has occurred, such as by way of example only and not by way of limitation, that the residual hearing of the recipient has changed and/or that a threshold level of the recipient has changed. Based on this evaluation, an alteration to the map and/or a new map can be implemented. This can be done automatically. It is noted that by automatically, this does not mean that the evaluation is necessarily automatic. In an exemplary embodiment, the recipient and/or a clinician can instruct the prosthesis or otherwise instruct the system that enables the evaluation to implement a method according to the teachings detailed herein. The execution of that method may be automatic, but the input that prompted the implementation of the method may not be automatic. (An exemplary algorithm involving the adjustment of a map and/or the utilization of a new map is described below.)

It is further noted that adjustments to the map and/or selection of a new map need not necessarily be a result of a physiological change of the recipient. In an exemplary embodiment, the evaluation of the data can be indicative of a change in the prosthesis, where there is utilitarian value with respect to adjusting the map and/or selecting a new map as a result of this change in the prosthesis.

Note also that an embodiment includes the ability to adjust a feature of the implantable component and/or the external component based on the evaluated data without doing so automatically. By way of example only and not by way of limitation, in an exemplary embodiment, the evaluation of the data and/or the data stored in the device or otherwise recorded in the device can be conveyed to the recipient and/or to a healthcare professional (as will be described in greater detail below), and based on the data, adjustments can be manually made to the prosthesis. In an exemplary embodiment, the prosthesis can provide an indication of the recipient (e.g., by evoking a hearing percept corresponding to this indication) that a given adjustment can be made to the prosthesis and/or that the data indicates a certain phenomenon occurring with the prosthesis. Accordingly, an exemplary embodiment can enable the recipient and/or the healthcare professional to provide input to the prosthesis so as to adjust the various features detailed herein. In an exemplary embodiment, the prosthesis can enable the recipient to incrementally adjust the location of the stimulation arrangement 350. Accordingly, in an exemplary embodiment, the recipient can be provided the data, and based on that data, the recipient can adjust the hearing prosthesis accordingly. In an exemplary embodiment, this can be done in an iterative manner, where subsequent data is also evaluated or otherwise provided to the recipient and further adjustments are made, etc. It is noted that while this exemplary embodiment has been explained in terms of the recipient implementing or otherwise controlling the adjustments to the prosthesis, in an exemplary embodiment, a healthcare professional can perform, initiate or otherwise implement the adjustments.

Corollary to this is that in an exemplary embodiment, the hearing prosthesis is configured to transcutaneously transmit the data based on the electrophysiological signals of the recipient resulting from the mechanical stimulation from the implantable component to the external component (whether that data is recorded or evaluated). By way of example, as detailed herein, such can be achieved via the transcutaneous inductance link between the implantable component and the external component. It is noted that in keeping with the broad meaning of data based on the signals, the raw signals can be transmitted or processed signals can be transmitted. Any arrangement of providing the data based on the electrophysiological signals to the external component can be utilized in at least some exemplary embodiments. Note further that the ability to transmit the data or otherwise communicate the data to the external component does not limit the prosthesis from being able to transmit the data to a component that is not part of the hearing prosthesis. In an exemplary embodiment, the recipient can go to a healthcare professional, where the healthcare professional downloads or otherwise extracts the recorded data that is recorded in implantable component. That said, in an alternate embodiment, the recipient can have a device that is configured to communicate with the implantable component beyond that which corresponds to the external component of the hearing prosthesis. In an exemplary embodiment, this can be a dedicated inductance coil relationship that is configured to communicate with a consumer and industrial products, such as by way of example, a laptop computer, and/or a personal computer, and/or a smart phone, and/or a so-called portable handheld device that is configured to receive the information from the implantable component.

That said, in at least some exemplary embodiments, the prosthesis is configured to transmit the data from the implantable component to the external component, which external component is also part of the hearing prosthesis. For example, in an exemplary embodiment, the raw electrophysiological signals and/or amplified raw electrophysiological signals are transmitted from the implantable component to the external component in the form of a BTE device or to another external component such as a consumer electronics product, as possibly modified to communicate with the implanted component (as is described by way of example below). The BTE device can record this data and/or can evaluate this data. In an exemplary embodiment, the BTE device records this data according to the various schemes detailed herein and/or other schemes that are usable to implement the teachings detailed herein. In an exemplary embodiment, the BTE device is configured to enable the transmission of this data to a remote location and/or to a consumer electronics product. For example, the BTE device can have an input/output port, such as a USB port, that enables communication with a personal computer. The recipient can download the data from the BTE device to the personal computer, and then email or otherwise transmit this data to a healthcare professional and a remote location. Alternatively, and/or in addition to this, this can be done automatically and/or semiautomatically, where the prosthesis is configured to download and/or upload information automatically. Indeed, in an exemplary embodiment, the BTE device can have a cellular phone communication and/or Wi-Fi communication capabilities. Some additional exemplary methods associated with the management, and/or evaluation, and/or the transmission of the data are described in greater detail below.

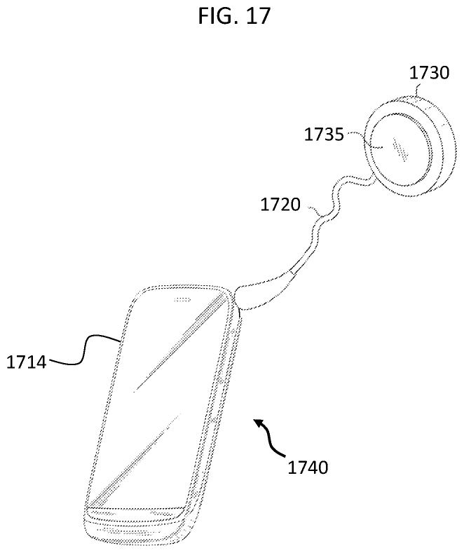

In this regard, FIG. 17 depicts an exemplary embodiment of a device 1740. FIG. 17 depicts a so-called smart phone or other portable handheld electronic device 1714. The smartphone 1714 is in signal communication with a headpiece 1730 via cable 1720. Headpiece 1730 is held against the skin of the recipient via a magnet 1735, which, in an exemplary embodiment, interacts with the magnet of the implantable component, by way of example only and not by way of limitation, when the implantable component is implanted via the skin of the recipient. In an exemplary embodiment, headpiece 1730 includes an inductance coil so as to enable communication with the implantable component. The smart phone 1714 communicates with the inductance coil via cable 1720. This exemplary embodiment can be utilized to download or otherwise retrieve any data stored in the implantable component. That said, in an alternate embodiment, the device 1740 can be utilized to download the data in real-time without any storage of data in the implantable component. Such can have utilitarian value with respect to an embodiment corresponding to a so-called totally implantable hearing prosthesis. Still further, in an exemplary embodiment, the smartphone 1714 can be configured to perform the evaluations detailed herein and/or analyze the data as detailed herein or otherwise process the data and develop configuration data that can be provided via the headpiece 1730 to the implantable component so as to adjust a feature thereof. That is, in an exemplary embodiment, the data based on the electrophysiological signals can be transferred to the smart phone 1714, and the smart phone 1713 can process or otherwise evaluate the data. Based on that evaluation, the smart phone 1714 can provide commands or otherwise data to the implantable component such that the implantable component, upon receipt of such data, adjusts a feature thereof. Corollary to this is that consistent with the embodiment where the changes to implantable component are executed not automatically but instead based on commands controlled by the recipient or the healthcare professional, the smart phone can present the results of the analysis and/or the data to the recipient and/or healthcare professional and, based on that data, the recipient can make adjustments to the prosthesis accordingly. Note further that in some exemplary embodiments, the smart phone 1714 is configured to electronically communicate the data to a remote location.

The embodiment of FIG. 17 is utilized for purposes of concept illustration. It is noted that other types of consumer products can be utilized in place of and/or in addition to the smart phone 1714, such as by way of example only and not by way of limitation, a personal computer, etc.

It is further noted that while the embodiment depicted in FIG. 17 utilizes a headpiece that is designed to communicate with the implantable component, alternatively and/or in addition to this, the embodiment of FIG. 17 can include a USB port, or the like, so as to enable communication with the external component. That said, in an alternate embodiment, the device 1740 is configured to communicate with the external component utilizing the headpiece 1730. In an exemplary embodiment, the recipient can place the headpiece 1730 against the corresponding headpiece of the external component so that the two components can communicate via a new inductance link that is completely external to the recipient, thus enabling the transfer of data between the two components. It is briefly noted that in an exemplary embodiment of such an exemplary embodiment can be configured so as to reverse the polarity of the magnet 1735 in the headpiece 1730 so that the headpiece 1730 is attracted to the headpiece of the external component. In an alternative embodiment, the headpiece 1730 is such that the inductance coil is positioned such that a transcutaneous inductance communication can occur utilizing one side of the headpiece and inductance communication with the external component can be executed using the other side of the headpiece (note that this is not necessarily simultaneously—this embodiment can include structure that simply enables such communication from different sides so as to address the magnet polarity issue).

Note further that in view of the various disclosures above, it is to be understood that an exemplary embodiment includes a hearing prosthesis system, comprising a direct acoustic cochlear stimulator (DACS) sub-system and an electrophysiology measurement sub-system (e.g., an electrocochleography measurement sub-system). In an exemplary embodiment, the sub-system that includes the actuator includes both direct intra cochlear acoustic stimulators and direct extra cochlear acoustic stimulators. Any disclosure herein of a DACS sub-system also supports a disclosure of a direct intra cochlear acoustic stimulator unless otherwise specified, while DACS is used per the standard meaning in the art at this time. In an exemplary embodiment, the DACS sub-system includes the middle ear implant stimulation arrangement 350 of FIG. 3 detailed above. Alternatively, and/or in addition to this, the DACS sub-system includes an inner ear mechanical stimulator. In an exemplary embodiment, the latter can correspond to an array of mechanical actuators that are arranged such that upon insertion into the cochlea, the mechanical actuators are tonotopically aligned with the pertinent portion of the cochlea. Movement of the actuators stimulates portions of the cochlea so as to evoke a hearing percept. The electrophysiology measurement sub-system can correspond to any of the test systems/sub-systems detailed herein and/or variations thereof.

In an exemplary embodiment of this exemplary embodiment, at least a portion of the DACS sub-system and at least a portion of the electrophysiology measurement sub-system are configured to be permanently implanted in a recipient. Accordingly, such an exemplary embodiment provides a hearing prosthesis system that can enable electrophysiology measurement without the utilization of a non-hearing prosthesis apparatus. (It is briefly noted that any device, system, and/or method detailed herein corresponds to such a disclosure of such that is without a non-hearing prosthesis device, although other embodiments include such with the addition of a non-hearing prosthesis device.)

Still further, in an exemplary embodiment, there is a hearing prosthesis system that includes a transceiver-stimulator unit (e.g., receiver unit 332 and stimulator unit 320 in combination). At least a portion of the direct acoustic cochlear stimulator sub-system and at least a portion of the electrophysiology measurement sub-system are part of the transceiver-stimulator unit. In this regard, by way of example only and not by way of limitation, such can correspond to the functional arrangement of FIG. 6, and/or FIG. 11, and/or FIG. 13, etc. In some such embodiments, the electrophysiology measurement sub-system includes a reference electrode and a measurement electrode at least in signal communication with the transceiver-stimulator unit. By “at least in signal communication with the transceiver stimulator unit,” it is meant that the electrodes are not necessarily supported or otherwise part of the transceiver stimulator unit. Instead, one or both the remote electrodes that are in communication with the unit via electrical lead to the like. That said, in an alternate embodiment, one or both of these electrodes are part of the transceiver stimulator unit, such as for example, reference electrode 380 as seen in FIG. 10. Concomitant with the embodiments detailed above, the transceiver-stimulator unit is configured to provide data indicative of input from the electrodes (output from the electrodes that corresponds to input to the unit) to an external component, which external component can correspond to a portion of the hearing prosthesis or which can correspond to another component, such as by way of example, the device of FIG. 17.

With respect to the embodiment of the mechanical cochlear implant, it is noted that an exemplary embodiment can include implementation of such where one or more measurement electrodes are supported by the mechanical cochlear implant such that the at least one measurement electrode is received in the cochlea when the mechanical cochlear implant is implanted therein. Accordingly, while the embodiments detailed above have been directed towards extra cochlear electrodes for utilization with the test system, some alternate embodiments include the utilization of one or more intracochlear measurement electrodes. Such embodiments can be combined with embodiments that utilize the extra cochlear electrode.

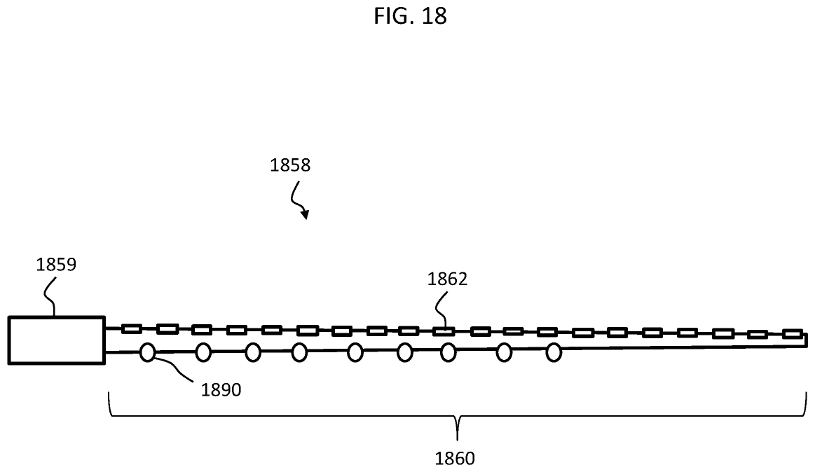

FIG. 18 presents an exemplary embodiment of a mechanical cochlear implant 1858 in the form of a direct intra cochlear acoustic stimulator. The implant 1858 includes an array 1860 of mechanical actuators 1862 and an extra cochlear portion 1859 that provides signal communication with the transceiver stimulator of the implantable component utilizing such implant. Also, the array 1860 also supports measurement electrodes 1890. The embodiment depicted in FIG. 18 includes a plurality of electrodes 1890. It is noted that in some alternate embodiments, only one measurement electrode 1890 is utilized in the intracochlear portion of the implant 1859. In an exemplary embodiment, the embodiment of FIG. 18 is presented with intra-cochlea acoustic and electrical stimulation where the basal region/low frequency regions of the cochlea are electrically stimulated and the apical region/higher frequency regions are acoustically stimulated by a mechanical device in the cochlea. In an exemplary embodiment, the electrodes 1890 can perform a dual function of stimulating the cochlea using electricity and also serving as a sense.

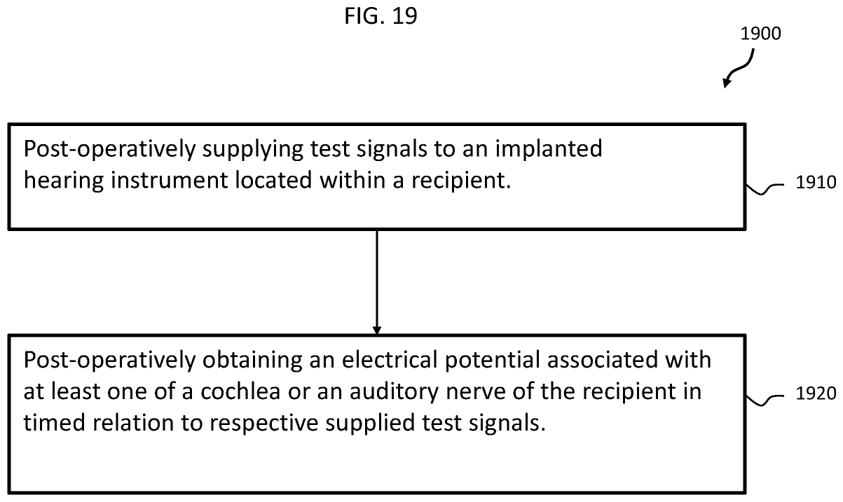

FIG. 19 depicts an exemplary an exemplary algorithm for an exemplary method 1900 according to an exemplary embodiment. Method 1900 includes method action 1910, which entails post-operatively supplying test signals to an implanted hearing instrument located within a recipient. By way of example only and not by way of limitation, this can entail supplying test signals to the stimulation arrangement 350. This can also or alternatively entail supplying test signals to the mechanical cochlear implant 1858 described above with respect to FIG. 18. This can also entail supplying test signals to an active transcutaneous bone conduction device, although in yet some other embodiments, this can entail supplying test signals to a passive transcutaneous bone conduction device or to a percutaneous bone conduction device. In this regard, some exemplary embodiments entail utilizing the electrophysiology measurements detailed herein and variations thereof to fit a percutaneous and/or a transcutaneous bone conduction device. In an exemplary embodiment, a measurement electrode can be placed in or near the cochlea, and the teachings detailed herein and/or variations thereof can be practiced for a bone conduction device. In an exemplary embodiment, the measurement electrodes can be part of the implantable component of the bone conduction device, while in other embodiments, the measurement electrodes can be part of a separate implant separate from the bone conduction device. Any arrangement that can enable the teachings detailed herein and/or variations thereof to be practiced with respect to a bone conduction device can be utilized in at least some exemplary embodiments. Method 1900 further includes method action 1920, which entails post-operatively obtaining an electrical potential associated with at least one of a cochlea or an auditory nerve of the recipient in timed relationship to the respective supply test signals. In embodiments according to the teachings detailed herein, method action 1920 is executed utilizing a component permanently implanted in the recipient. By “permanently implanted in the recipient,” it is meant a device that is implanted such that it is not intended to be removed from the recipient except in the event of failure of the component, incompatibility of component with the recipient, obsolescence of the component, etc. This is opposed to a diagnostic or test device that is implanted for purposes of performing testing with the expectation that such component will be removed within a reasonable period after the testing. In an exemplary embodiment, the component that is permanently implanted in the recipient is a component that has been implanted in the recipient for at least 30, 60, 90, 180, 270, 360, 500, 750, 1000 days, or more. In this regard, in an exemplary embodiment, the actions of method 1900 are executed after a period of time from the operation that implanted the component corresponding to the aforementioned temporal periods. That said, it is noted that method 1900 can be executed the very next day or even hours after the operation. In an exemplary embodiment, the method 1900 is executed within a few hours of the medical personnel “closing” the incisions in the recipient that enabled the implantation of the implanted hearing instrument and/or the component permanently implanted in the recipient.

It is noted that in an exemplary embodiment, the components permanently implanted in the recipient can correspond to one or more of the aforementioned measurement electrodes and/or the reference electrode detailed herein. Accordingly, the components permanently implanted in the recipient can be part of the implanted hearing instrument, and thus in this exemplary embodiment, the implanted hearing instrument is a hearing instrument that is permanently implanted in the recipient.

In an exemplary embodiment, method action 1920 is executed entirely with an implanted component of the implanted hearing instrument. For example, method action 1920 can be executed utilizing the embodiment of FIG. 6 detailed above. Note that in an exemplary embodiment, the action of supplying the test signal (action 1910) is executed using an external component of a hearing prosthesis of which the implanted hearing instrument is apart and using the implanted hearing instrument. As with method action 1920, this action can be executed utilizing the embodiment of FIG. 6. This embodiment can be executed utilizing the embodiment of FIGS. 11 and/or 13.

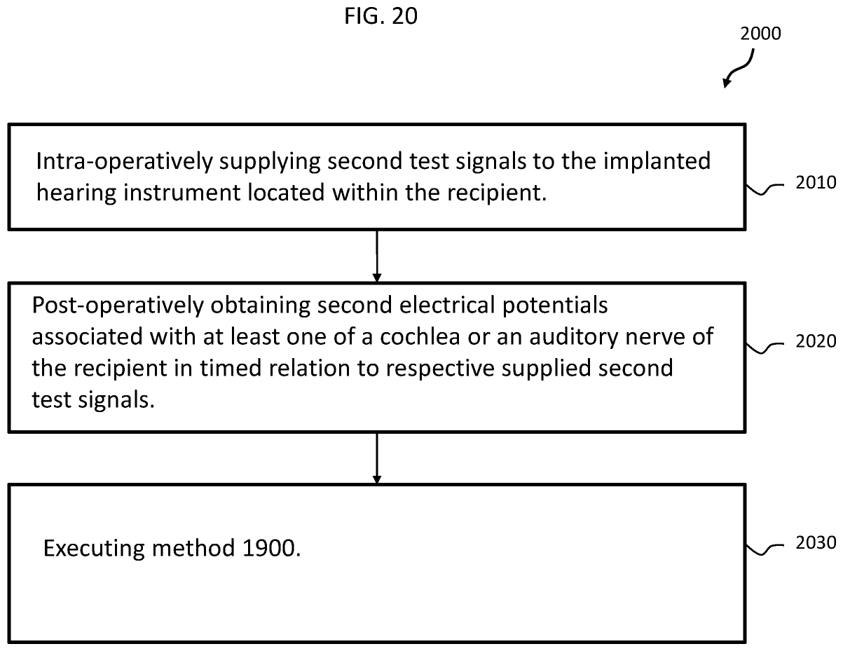

FIG. 20 depicts an exemplary algorithm for another exemplary method, method 2000. Method 2000 is a method that is executed prior to method 1900. It is briefly noted at this time that the nomenclature “second” is utilized for purposes of naming convention only, and does not represent primacy and/or the temporal order in which these method actions are executed.

As can be seen, method 2000 includes method action 2010 which entails intra-operatively supplying second test signals to the implanted hearing instrument located within the recipient. Method 2000 further includes method action 2020, which entails intra-operatively obtaining the second electrical potentials associated with at least one of the cochlea or the auditory nerve of the recipient in timed relation to the respective supplied second test signals. As with method action 1920, detailed above, method action 2020 is executed using the component permanently implanted in the recipient. Still with reference to FIG. 20, it can be seen that method 2000 further includes method action 2030, which entails executing method 1900, consistent with the fact that method 2000 is a method that is executed intra-operatively (i.e., during the surgical procedure in which the component permanently implanted in the recipient is implanted in the recipient and/or in which the implanted hearing instrument is implanted in the recipient), and method 1900 is a method that is executed after the operation.

Accordingly, in view of method 2000, it is to be understood that the teachings detailed herein and/or variations thereof have utilitarian value with respect to both post-operative scenarios and intra-operative scenarios.

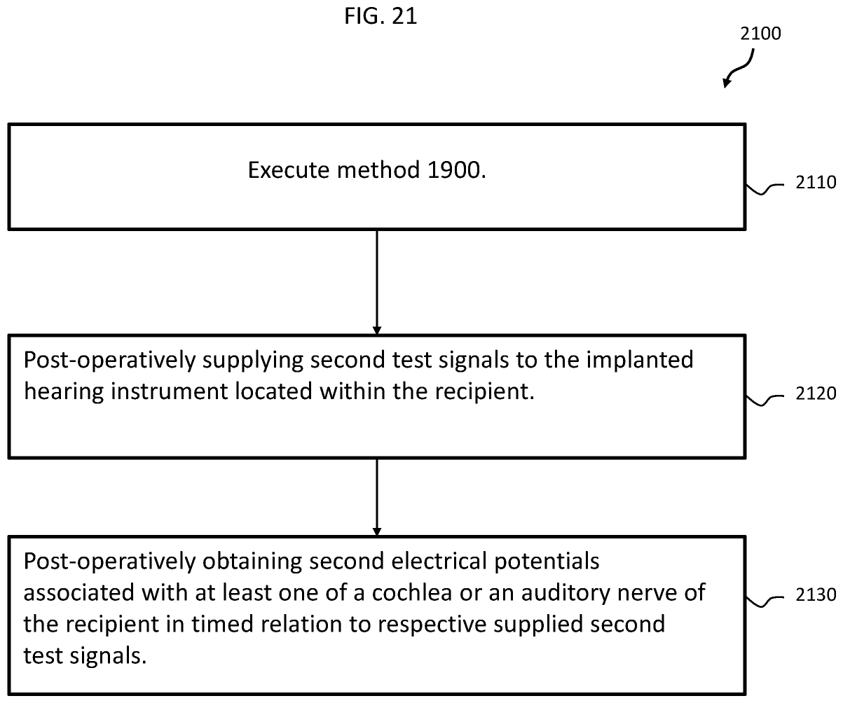

It is further noted that method 1900 is a method that can be repeated after the method is executed. In this regard, FIG. 21 presents an algorithm for another exemplary method, method 2100, which includes method action 2110, which entails executing method 1900. Subsequent method action 2110, method 2100 includes executing method action 2120, which entails post-operatively supplying second test signals to the implanted hearing instrument located within the recipient, and method action 2130, which entails post-operatively obtaining second electrical potentials associated with at least one of a cochlea or an auditory nerve of the recipient in timed relation to the respective supplied second test signals. Corollary to method 2100 is that method 1900 can be repeated frequently. In this regard, an exemplary method entails repeating method 1900 over the course of days, weeks, months, or years, at least once a day, at least once a week, at least once a month, at least once every two months, at least once every three months, at least once every four months, at least once every five months, at least once every six months, at least once every nine months, at least once every 12 months, at least once every 15 months, or at least once every 18 months. In an exemplary embodiment, method action 1900 can be executed somewhat continuously in that it could be executed every hour.

In view of the teachings detailed above associated with FIGS. 6, 11, and 13, it is to be understood that in an exemplary embodiment, the methods detailed herein and/or variations thereof can be executed such that the actions of supplying the test signals and obtaining the electrical potentials are executed utilizing a hearing prosthesis of which the component permanently implanted in the recipient is a part.

It is further noted that in an exemplary embodiment, method 1900 and method 2100 can be executed such that the actions of supplying test signals and obtaining the electrical potentials are executed while the recipient is performing tasks corresponding to daily living of the recipient. That said, in an alternate embodiment, these actions are executed in close temporal proximity to the recipient performing tasks corresponding to daily living. Still further, in an exemplary embodiment, these actions can be executed while the recipient is sleeping. In some exemplary embodiments, these actions are implemented or otherwise initiated in an automatic fashion by the hearing prosthesis. In this regard, in an exemplary embodiment, there are hearing prostheses according to the teachings detailed herein that are configured to enable one, or more, or all of the method actions detailed herein and/or variations thereof to be executed in an automated fashion according to a schedule and/or according to an occurrence of a given scenario. With respect to the former, in an exemplary embodiment, there is a hearing prosthesis that is configured to automatically initiate method 1900 whenever the prosthesis determines that the recipient is sleeping. Alternatively, and/or in addition to this, there is a hearing prosthesis that is configured to automatically initiate method 1900 whenever the prosthesis determines that the recipient is in a silent environment. Alternatively, and/or in addition to this, there is a hearing prosthesis that is configured to automatically initiate method 1900 at a certain time of day (including a certain time of night). Note further, by way of example only and not by way of limitation, in an exemplary embodiment there is a hearing prosthesis that is configured to receive a command from a remote device so as to initiate method 1900. Also, in an exemplary embodiment, there is a prosthesis that is configured so that the recipient can initiate method 1900 himself or herself.

It is briefly noted that with respect to actions executed while the recipient is sleeping, in some embodiments, the stimulation that is provided to the tissue of the recipient entails stimulating an ear component of the recipient with energy that is at a sub hearing threshold and/or a supra hearing threshold. That is, the energy does not evoke a hearing percept that is recognizable or otherwise noticeable to the recipient. Such can have utilitarian value with respect to executing at least some of the method actions detailed herein while the recipient is sleeping. That said, the sub threshold and/or supra threshold stimulation need not necessarily be executed only while the recipient is sleeping. Such can be executed at any time where such has utilitarian value. Note further, that some embodiments can be executed utilizing stimulation that results in a hearing percept while the recipient is sleeping.

By way of example only and not by way of limitation, sub threshold and/or supra threshold stimulation can entail stimulating the tissue at frequencies below and above, respectively, the recipient's hearing (including residual hearing). Still further by way of example only and not by way limitation, sub threshold stimulation can entail stimulation at a magnitude that is below the recipients ability to hear. Indeed, in an exemplary embodiment, automated regularly scheduled or non-scheduled or non-regularly scheduled post-operative checks can be performed. There can be utilitarian value with respect to performing these at sub-threshold levels to avoid disturbing the recipient or otherwise distracting the recipient. Indeed, in an exemplary embodiment, the post-operative testing (e.g., the automated testing), is performed in a quiet environment, where in at least some instances, the recipient is sleeping. In any event, in an exemplary embodiment, the various method actions are executed in a manner that does not interfere with regular hearing, at least to any meaningful extent. Any arrangement that can enable the sub or supra threshold stimulation can be utilized in at least some exemplary embodiments.

Embodiments utilizing sub and/or supra threshold stimulation can have utilitarian value with respect to implement in some or all of the methods detailed herein without the recipient consciously recognizing that the methods are executed or otherwise without distracting the recipient. Accordingly, in an exemplary embodiment, there is one or more of the method actions detailed herein that is executed without the recipient knowing, or at least without the recipient recognizing directly that the method actions are being executed (the recipient may recognize such indirectly because the recipient knows that at a given time a day, the methods are executed, or the prosthesis may indicate to the recipient that such is the case). Accordingly, an exemplary embodiment entails executing one or more of the methods detailed herein and/or variations thereof while the recipient is fully conscious and while the recipient has an alert level of cognitive capacity where the execution of the actions does not directly reveal to the recipient that such actions have occurred (again, the recipient may know or not know that the method actions have been executed or are being executed for other reasons). It is briefly noted that in an exemplary embodiment, some or all of the fitting actions detailed herein are executed without utilizing behavioral measurements. Corollary to this is that in at least some exemplary embodiments, the fitting methods detailed herein and variations thereof are executed not only utilizing behavioral measurements (e.g., the teachings detailed herein and/or variations thereof can be utilized in conjunction with behavioral measurements).

With respect to performing tasks of daily living, it is meant that method 1900 can be executed while the recipient is going about his or her daily business. That is, method 1900 does not require the recipient to be in or otherwise be associated with a specific testing environment (e.g., be in a testing center or be in a sound controlled environment, etc., although some implementations of the teachings detailed herein can certainly be executed in a test center or in a sound controlled environment) during the execution of method 1900.

As noted above, method 1900 can be executed while the recipient is conducting tasks associated with daily living. In an exemplary embodiment, method 1900 can be executed while the recipient is driving in a car. In an exemplary embodiment, method 1900 can be executed while the recipient is reading. In an exemplary embodiment, method 1900 can be executed while the recipient is typing. In an exemplary embodiment, method 1900 can be executed while the recipient is exercising or playing a sport. In an exemplary embodiment, method 1900 can be executed while the recipient is watching television or playing a videogame. In an exemplary embodiment, method 1900 can be executed while the recipient is taking a shower (e.g., in the case of a totally implantable hearing prosthesis). In an exemplary embodiment, method 1900 is executed without the recipient being at a healthcare location. In an exemplary embodiment, method 1900 is executed without the recipient being in real-time communication with a healthcare location or a healthcare provider or an agent thereof.

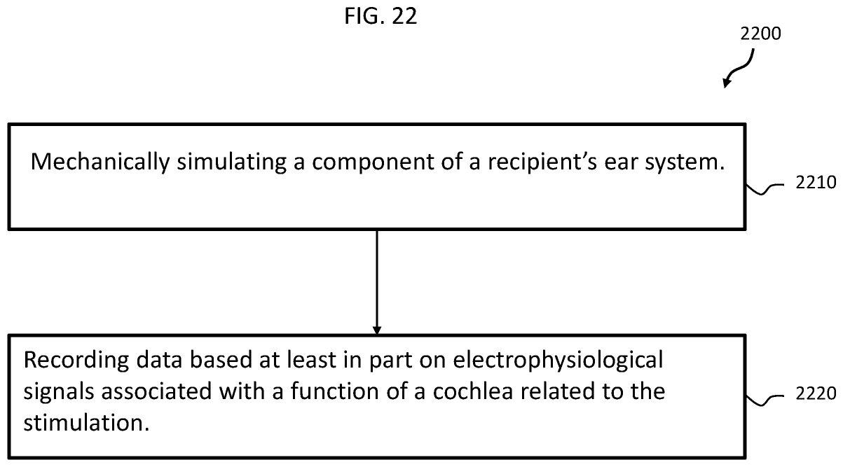

FIG. 22 depicts another algorithm for an exemplary method, method 2200. Method 2200 includes method action 2210, which entails mechanically stimulating a component of the recipient's ear system. In an exemplary embodiment, this can be executed utilizing any of the prostheses detailed herein and/or variations thereof. Method 2200 further includes method action 2220, which entails recording data based at least in part one electrophysiological signals associated with a function of a cochlea related to the stimulation. In an embodiment of method 2200, action 2210 and action 2220 are executed entirely using a device permanently implanted in a recipient. In an exemplary embodiment, by way of example only, this can be executed utilizing the test sub-unit 1361 in general, and device 1505 in particular, which, as detailed above, can be a memory unit. Any arrangement of recording the data can be utilized in at least some exemplary embodiments.

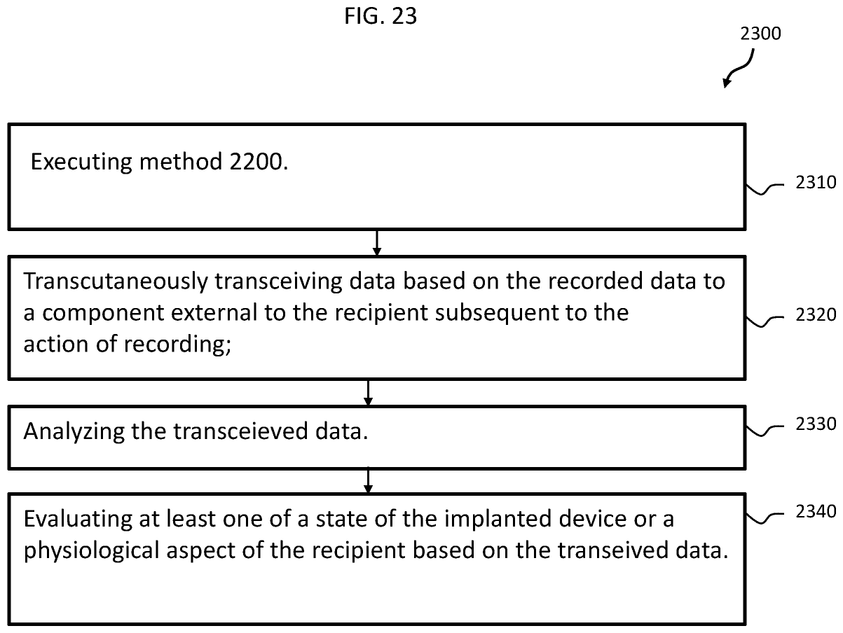

FIG. 23 depicts another algorithm according to an exemplary embodiment for a method, method 2300. Method 2300 includes method action 2310, which entails executing method 2200. Method 2300 further includes method action 2320, which entails transcutaneously transceiving data based on the recorded data to a component external to the recipient subsequent to the action of recording. By “transceiving,” it is meant that one or both of the following occurs: the implantable component transmits the data or the external component extracts the data. In this regard, in an exemplary embodiment, method 2320 can be executed with an external component that has the capability of reading the memory of the implantable component, and thus as a technical matter, the internal component might not necessarily be considered to transmit the data (although there are those in the art who would hold the position that such also constitutes transmitting—it is noted at this time that unless otherwise stated, any disclosure of an action of transmitting or the ability to transmit data also corresponds to a disclosure of an ability of the “receiving” device and the action of the “receiving” device to read the data without an affirmative action on the part of the component that is in possession of the data).

In an exemplary embodiment, method action 2320 can be executed utilizing the external component of the hearing prostheses detailed herein and/or variations thereof, such as external component 1142 or external component 1342 or any of the other external components detailed herein that have the ability to execute method action 2320. In an exemplary embodiment, method action 2320 can also be executed utilizing the device of FIG. 17 or the related devices associated therewith or any other component that can enable method action 2320 to be executed.

Method 2300 further includes method action 2330, which entails analyzing the transceived data. In an exemplary embodiment, this can correspond to any of the teachings detailed herein associated with analyzing, which details are also provided in greater detail below. In an exemplary embodiment, method action 2330 can be executed using the external component of the hearing prosthesis, or can be utilized using another type of external component, such as a remote consumer electronics device. It is further noted that method 2330 can be executed remotely from the recipient. As detailed above, some exemplary embodiments enable the transmission of the data to a remote location, such as a healthcare provider or the like, where method action 2330 can be executed. That said, method action 2330 can be executed automatically by the hearing prosthesis and/or by the consumer electronics product in possession of the recipient.

Method 2300 further includes method action 2340, which entails evaluating at least one of a state of the implanted device or a physiological aspect of the recipient based on the transceived data. In this regard, a state of the implanted device can correspond to the location of the implanted device and/or the amount of loading of the implanted device. In an exemplary embodiment, the state of the implanted device can correspond to the resonant frequency of the implanted device. Any of the features associated with the implantable device detailed herein that can be adjusted can be included in the evaluation of the state of the implantable device. That said, the state of the implantable device can also include features that are not adjustable as well. Indeed, the evaluation of method 2340 can entail an evaluation where the state of the implanted device is broken.

In an exemplary embodiment, the methods detailed herein and/or variations thereof can enable the measurement of the cochlea coupling efficiency associated with the hearing prosthesis. In an exemplary embodiment, tissue stimulation can occur at a variety of different frequencies so as to develop a base of data to perform analysis. Based on the obtained electrical potentials and/or the obtained electrophysiological signals associated with a function of the cochlea related to the stimulation, and in correlation with data pertaining to the stimulation, an exemplary embodiment entails measuring or otherwise determining or otherwise evaluating the cochlea coupling efficiency of the hearing prosthesis.

In at least some exemplary scenarios, the coupling between the actuator and the ossicles with the coupling between the actuator and the cochlea, etc., and a greater otherwise change over time. This is sometimes referred to coupling deterioration. In an exemplary embodiment, the teachings detailed herein can be utilized to identify the occurrence of coupling deterioration. This can be done utilizing the methods detailed herein and/or variations thereof. In an exemplary embodiment, the implant can execute one or more of the various actions detailed herein automatically, or such can be done upon an initiation from a control unit remote from the implant, to evaluate the coupling between the actuator and the structure of the recipient. In an exemplary embodiment, based on the measurements obtained, an evaluation can be made as to whether or not the coupling has deteriorated and/or an amount of deterioration. In evaluation can further be made as to whether or not an adjustment to the implant should be made, which adjustment could be performed via a mildly invasive surgical procedure, and/or could be performed automatically via the implant. With respect to the former, this can entail accessing the actuator and moving the actuator to a new location to account for this deterioration. With respect to the latter, this can entail adjusting the location of the actuator utilizing the various components of the implant to move the actuator to a new location that at least partially compensates for this deterioration. Still with respect to the latter, this can also entail adjusting a gain of the like of the implant so as to compensate for this deterioration.

With regards to a physiological aspect of the recipient based on the transceived data, such can entail an evaluation of the recipient's residual hearing and/or an evaluation of the recipient's nervous system with respect to the hearing system and/or an evaluation of the threshold and/or comfort levels associated with the recipient vis-à-vis the artificial hearing percepts resulting from activation of the implanted hearing prosthesis. Any physiological feature of the recipient that can be evaluated utilizing the teachings detailed herein and/or variations thereof can be included in method action 2340.

Consistent with the teachings detailed above with respect to sub and super threshold simulation, an exemplary embodiment entails executing method action 2210 such that the mechanical stimulation of the component of the recipient's ear system occurs at a sub or threshold level.

It is noted that while the embodiment of FIG. 23 details the action of transcutaneously transceiving the data, in an exemplary embodiment, method action 2320 can be skipped, and the implantable component can perform method actions 2330 and/or 2340. Note further that method action 2330 can be executed by the implantable component (based on the recorded data—not the transceived data), and then method action 2320 can be executed, where the transceived data corresponds to data resulting from the analysis of the recorded data executed by the implantable component. Note further that in exemplary embodiment, instead of recording data in method 2200, the data is analyzed and evaluated by the hearing prosthesis in real time.

Some exemplary embodiments further include a method that entails executing method 2200, and then analyzing the recorded data (although, as noted above, in some exemplary embodiments, it is not necessary to record the data, and the data is simply analyzed, or alternatively, the data is analyzed and the results of that analysis are recorded). This exemplary method further includes determining that at least one of a physiological change in the recipient or a status change of the device that is permanently implanted in the recipient has occurred relative to a baseline reference based on the analysis. By way of example only and not by way limitation, the baseline can be subjective to the particular recipient. In an exemplary embodiment, within a few days and/or a few weeks, or after the utilitarian healing time after implantation of the prosthesis, a series of tests can be run on the recipient. Such tests can entail executing one or more or all of the method actions detailed herein and/or variations thereof automatically and/or under the control of a healthcare professional, etc. Such can also entail executing other types of tests. Any testing that can develop a baseline for this method can be utilized in at least some exemplary embodiments. That said, in some alternative embodiments, the baseline can be a statistical based baseline. That is, the baseline can correspond to what “should be” the case for a recipient falling within a given human factors category. Any baseline that can have utility can be utilized in at least some exemplary embodiments.

In an exemplary embodiment, the action of determining that at least one of a physiological change in the recipient or a status change of the devices occurred relative to a baseline reference is executed based entirely on the recorded data.

In an exemplary embodiment, the action of determining that at least one of a physiological change in the recipient or a status change of the device has occurred can entail determining any of the physiological changes and/or status changes detailed herein. With respect to the former, such can entail determining that the recipient's residual hearing has changed (increased and/or decreased, both with respect to overall and with respect to specific frequencies and/or with respect to threshold and/or comfort levels etc.). With respect to the latter, this can entail a determination that the resonant frequency of the implantable device has changed and/or that the device has moved, etc.

In an exemplary embodiment, an exemplary method entails, upon such a determination that the physiological change or the status change of device has occurred, an alarm can be issued to the recipient based on this determination. In an exemplary embodiment, this can be executed automatically by the hearing prosthesis. Such can be done via an electronic communication to a remote device by the hearing prosthesis at, for example, the first instance where this remote device is in communication with the hearing prosthesis. Such can also be done via communication with the recipient directly from the prosthesis. For example, the prosthesis can provide an audio indication to the recipient via evoking an artificial hearing percept that something has changed. Alternatively, and/or in addition to this, a light indicator or the like on the hearing prosthesis can provide such indication.

The indication to the recipient can occur in real time (i.e., right away) upon the determination, or can occur during a status period subsequent to the determination. For example, once a week and/or once a month etc., the recipient might evaluate the performance of the hearing prosthesis so as to make adjustments of the like as part of a semi-scheduled routine. It can be during that evaluation that the recipient is provided the alarm, which alarm can entail an indication in a text document evaluation report of the hearing that the recipient utilizes during the evaluation period.

Any type of alarm or any alarm regime that can enable the teachings detailed herein or otherwise have utilitarian value can be utilized in at least some exemplary embodiments.

It is noted that some exemplary methods detailed herein and some exemplary devices detailed herein enable completely objective testing of a recipient (i.e., the testing can be 100% objective). Such can have utilitarian value with respect to implementing the methods detailed herein and/or variations thereof with respect to a child and/or an infant or otherwise with respect to a pre-pubescent recipient. In this regard, the various determinations and/or evaluations detailed herein and variations thereof can be executed without subjective feedback from the recipient. That is, in an exemplary embodiment, because the ECOG and/or the NRT data can be acquired without any communication from the recipient, the teachings detailed herein can be executed without subjective input from the recipient. That said, in some alternate embodiments, the teachings detailed herein are combined with subjective input from the recipient.

Exemplary embodiments that have utilitarian value with respect to young children and/or infants or otherwise with respect to pre-pubescent recipients, entail tissue stimulation by the hearing prosthesis to evoke a neural reaction via the application of a pure or complex signal (e.g., a non-sinus ordeal signal such as a chirp). In this regard, signal generator 606 can be a signal generator configured to output such signals. That said, in some alternate embodiments, the signal generator 606 is not utilized. Instead, speech and/or noise-like input is utilized as a basis for the tissue stimulation.

In view of the above, an exemplary embodiment entails measuring of the cochlea coupling efficiency at a plurality of different frequencies, assessing a residual hearing of the recipient, and based on the measuring in the assessment, determining if these measurements are deviating away from a clinical baseline measured in a clinic or otherwise measured at a prior point in time. Based on such, an alarm or otherwise indication can be provided to the recipient and/or the healthcare professional in the event that the measurements are outside of an acceptable clinical limit. Alternatively and/or in addition to this, these measurements and the analysis can be data logged or the like.

An exemplary embodiment can entail utilizing the teachings detailed herein to fit a hearing prosthesis to a given recipient. In an exemplary embodiment, the data based on the electrophysiological signals can be analyzed to determine a proper fitting or otherwise a utilitarian fitting of the hearing prosthesis. In an exemplary embodiment, this can be done remotely from a healthcare facility or the like. That said, in an alternative embodiment, communications technologies can be utilized such that the fitting can be executed by a healthcare professional remotely from the recipient. In an exemplary embodiment, the prosthesis itself or a fitting system utilized in conjunction with the prosthesis can be configured for full automation of the fitting of the prosthesis. In an exemplary embodiment, this can be executed utilizing the data based on the electrophysiological signals and no other data (aside from the correlated stimulation data). Accordingly, in an exemplary embodiment, such can entail a fully objective fitting method. In an exemplary embodiment, this can be implemented in a fully automatic fashion.

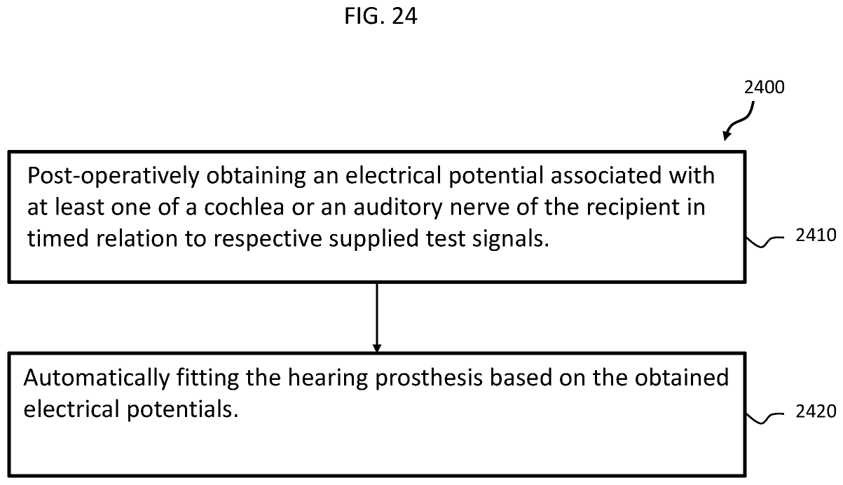

Accordingly, FIG. 24 presents an exemplary algorithm for an exemplary method 2400. Method 2400 includes method action 2410, which entails post operatively obtaining an electrical potential associated with at least one of a cochlea or an auditory nerve of the recipient in time relation to respective supplied test signals. In an exemplary embodiment, this can be executed utilizing any of the devices detailed herein. Method 2400 further includes method action 2420, which entails automatically fitting the hearing prosthesis based on the obtained electrical potentials. In an exemplary embodiment, method 2400 can be performed entirely remotely from a healthcare facility. In an exemplary embodiment, method 2400 can be executed by placing the hearing prosthesis into signal communication with a consumer electronics product, such as a personal computer or the like, where the personal computer (or other consumer electronics product, such as by way of example, a smart phone) contains thereon software or otherwise a program product to implement the automatic fitting based on the obtained electrical potentials. More specifically, in an exemplary embodiment, there is a non-transitory computer readable medium having recorded thereon, a computer program for executing a method, the program including code for automatically executing one or more of the method actions detailed herein and variations thereof. Still further, the program can include additional code for automatically executing one or more other method actions detailed herein.

Further, it is to be understood that in at least some exemplary embodiments, the teachings detailed herein can be utilized to avoid or otherwise mitigate a need for prescriptive fitting models/prescriptive fitting methods (e.g., those based on a population averages of hearing losses). Accordingly, in an exemplary embodiment, some or all of the method actions detailed herein can be executed without utilizing a prescriptive fitting model/methods. That said, in an alternate embodiment, the teachings detailed herein can be utilized in conjunction with a prescriptive fitting models/method. In this regard, it is to be understood that in at least some embodiments, the methods detailed herein can be executed so as to obtain a customized personalized fitting for the recipient, as opposed to fitting the hearing prosthesis based on statistical likelihoods. Still, in at least some embodiments, prescriptive methods and models can be utilized, but the fitting is primarily personalized to a specific recipient. In some embodiments, there is a system that enables the measurement of air conduction loss (e.g. via an audiogram) and the measurement of the coupling of the DACS, and the measurement of the dynamic range of the implant from sub to supra threshold. Thus, in an exemplary embodiment, the teachings detailed herein can be utilized to free an audiologist from utilizing or at least primarily relying one prescriptive based fitting methods to fit the implant.

More particularly, in an exemplary embodiment, there is a consumer electronics product application that enables the user to fit the hearing prosthesis at home or otherwise away from the clinical care location. In an exemplary embodiment, such application enables the fitting to be executed without having any communication with the clinical care facility or a healthcare provider, or otherwise can be done autonomously without having input there from. In this regard, the consumer electronics product enables recipient directed fitting of the hearing prosthesis remote from a healthcare clinic. (It is noted that in some alternate embodiments, the hearing prosthesis itself, without the consumer electronics product enables such recipient directed fitting.)

Thus, embodiments include non-transitory computer-readable media having recorded thereon, a computer program for executing one or more or any of the method actions detailed herein. Indeed, in an exemplary embodiment, there is a non-transitory computer-readable media having recorded thereon, a computer program for executing at least a portion of a method detailed herein and/or variations thereof. In an exemplary embodiment, the computer program is a program for automated and/or semiautomated recipient directed fitting of the hearing prosthesis.

In an exemplary embodiment, the media is such that at least some of the method actions detailed herein can be executed automatically. In an exemplary embodiment, the media is such that at least some of the method actions can be initiated automatically.

In yet another exemplary embodiment, the media further includes code for prompting a user to input subjective inputs into the consumer electronics product (and/or the hearing prosthesis). In this regard, embodiments include a combination of subjective and objective input into the consumer electronics product so as to fit the hearing prostheses.

In an alternative embodiment, semi-automated fitting of the hearing prosthesis can be implemented using some of the teachings detailed herein. Such can be achieved by combining objective test results with subjective test results. In this regard, some exemplary embodiments entail a consumer electronics product that is configured so as to automatically verify an acoustic fit derived by the recipient using objective measures of the system, and, in some other embodiments, also enable the adjustment or otherwise setting of fitting parameters based on subjective input.

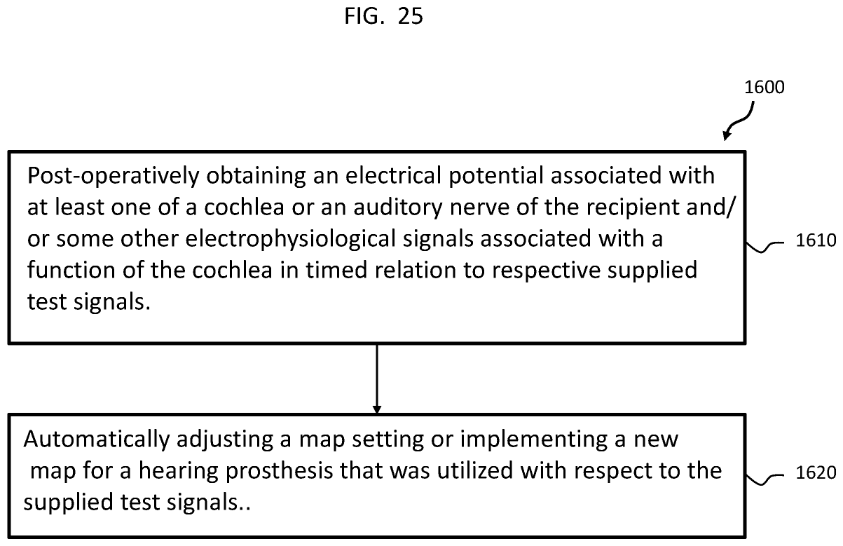

As noted above, embodiments entail making adjustments to the hearing prosthesis based on the data pertaining to the electrophysiological signals obtained by the implanted components. In this regard, an exemplary embodiment includes the algorithm presented in FIG. 25. In particular, this algorithm represents a method 1600. Method 1600 includes method action 1610, which entails, by way of example only and not by way limitation, post-operatively obtaining an electrical potential or some other electrophysiological signals associated with a function of the cochlea associated with at least one of the cochlea or and auditory nerve of the recipient in timed relation to respective supplied test signals.

Method 1600 further includes method action 1620, which entails automatically adjusting a map setting or implementing a new map for a hearing prosthesis that was utilized with respect to the supplied test signals. It is further noted that while the method 1600 is presented as having two actions, additional actions can be provided or otherwise implemented in such a method. By way of example only and not by way of limitation, an exemplary method 1600 further includes analyzing the obtained electrical potentials/signals, etc. By way of example only and not by way of limitation, such can entail analyzing the obtained data to determine a so-called cochlear coupling efficiency of the hearing prosthesis and/or analyzing the obtained data to determine a physiological feature of the recipient. Based on this analysis, the precise actions involved in executing method action 1620 can be determined.

Method 1600 can be executed while the recipient is going about tasks associated with daily living. As with at least most of the methods detailed herein, the actions associated with method 1600 can be executed remotely from a health care facility and/or without the assistance of a healthcare professional.

Note further that while method 1600 focuses on adjusting a map setting or implementing a new map for the hearing prosthesis, as noted above, alternate embodiments of adjusting a feature of the hearing prosthesis can include moving the position of the stimulation apparatus, etc. Such too can be performed automatically. That said, an exemplary embodiment entails executing method action 1620 in a non-automated fashion.

Some exemplary embodiments of the hearing prostheses detailed herein and/or the consumer electronics devices detailed herein can be configured so as to determine acoustic hearing profiles in recipients. In an exemplary embodiment, such are configured to automatically detect a threshold based on data based on the electrophysiological signals. Such can also be configured to characterize loudness growth and/or identify maximum comfort levels based on the electrophysiological signals and/or the data based on the electrophysiological signals. In some instances, the devices are configured to do so automatically.

As is to be understood from the above, some exemplary embodiments entail the evaluation of data (e.g., data based on the electrophysiological signals obtained by the implanted device, etc.). In an exemplary embodiment, evaluation can entail conclusory evaluation that results in a conclusion as to what the data indicates (e.g., that the stimulation arrangement has moved since implantation, etc.). Still further, in an exemplary embodiment, evaluation can entail non-conclusory evaluation that does not result in a conclusion as to what the data indicates, but instead results data that is utilized to make an adjustment or otherwise make a determination. For example, an evaluation can entail comparing data to that of a lookup table, and based on the comparison, taking an action without ever determining why that action is being taken.

It is noted that any device and/or system detailed herein also corresponds to a disclosure of a method of operating that device and/or using that device. Furthermore, any device and/or system detailed herein also corresponds to a disclosure of manufacturing or otherwise providing that device and/or system. Corollary to this is that any disclosure of a method herein corresponds to a disclosure of a device and/or system of implementing that method and/or a program product for implementing that method on a computer apparatus. Still further, any functionality of any device and/or system detailed herein corresponds to a method of taking action to achieve such functionality.

Any teaching of any embodiment detailed herein can be combined with one or more of other teachings of other embodiments detailed herein, providing that the art enables such, unless otherwise specified. It is further noted that any particular one or more teachings detailed herein can be omitted from an embodiment when implementing some exemplary embodiments.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method, comprising:
supplying signals to a hearing instrument attached to a recipient; and
obtaining data based at least in part on electrical properties associated with a function of an organ of a human associated with hearing.

2. The method of claim 1, wherein:
the action of supplying signals corresponds to supplying test signals to the hearing instrument, wherein the hearing instrument is an implanted hearing instrument located within a recipient.

3. The method of claim 2, wherein:
the action of obtaining data based at least in part on electrical properties includes obtaining an electrical potential associated with at least one of a cochlea, an auditory nerve, a brain stem or a brain of the recipient in timed relation to respective supplied test signals, wherein
the action of obtaining the electrical potential is executed using a component permanently implanted in the recipient.

4. The method of claim 1, wherein:
the action of obtaining data includes recording data based at least in part on electrophysiological signals associated with a function of a cochlea related to the stimulation.

5. The method of claim 3, wherein:
the action of obtaining data and the action of supplying signals is executed post-operatively.

6. The method of claim 3, wherein:
the action of obtaining data and the action of supplying signals is executed during an operation implanting a component of the hearing instrument.

7. The method of claim 5, wherein:
the action of obtaining the electrical potential is executed entirely with an implanted component of the implanted hearing instrument.

8. The method of claim 4, wherein:
the action of supplying signals to the hearing instrument results in an action of directly or indirectly mechanically simulating a component of the recipient's ear system.

9. The method of claim 5, further comprising:
intra-operatively supplying second test signals to the implanted hearing instrument located within the recipient; and
intra-operatively obtaining second electrical potentials associated with at least one of the cochlea or the auditory nerve of the recipient in timed relation to respective supplied second test signals, wherein
the action of obtaining the second electrical potential is executed using the component permanently implanted in the recipient.

10. The method of claim 3, wherein:
the actions of obtaining data and the action of supplying signals are executed at least a year after implantation of the implanted hearing instrument.

11. The method of claim 5, further comprising:
at least one of semi-automatically fitting or fully automatically fitting the implanted hearing prosthesis based on the obtained electrical potentials.

12. The method of claim 8, further comprising:
the action of stimulating and the action of recording the signals is executed entirely using a device permanently implanted in a recipient.

13. The method of claim 12, further comprising:
fitting the device permanently implanted in the recipient based on the recorded data.

14. The method of claim 5, wherein:
the actions of supplying test signals and obtaining the electrical potential is executed entirely using a hearing prosthesis of which the component permanently implanted in the recipient is a part.

15. The method of claim 12, wherein:
the action of mechanically stimulating the component of the recipient's ear system includes stimulating the component with energy that is at a sub hearing threshold and/or a supra hearing threshold.

16. The method of claim 15, wherein:
the action of mechanically stimulating the component of the recipient's ear system at the thresholds occurs automatically by the implanted device.

17. The method of claim 12, further comprising:
analyzing the recorded data; and
evaluating a change in the recipient's hearing based on the analysis.

18. The method of claim 12, further comprising:
analyzing the recorded data;
determining that at least one of a physiological change in the recipient or a status change of the device has occurred relative to a baseline reference based on the analysis.

19. The method of claim 12, further comprising:
analyzing the recorded data;
determining that at least one of a physiological change in the recipient or a status change of the device has occurred relative to a baseline reference based on the analysis; and
issuing an alarm to the recipient based on the determination.

20. The method of claim 5, wherein:
the actions of supplying test signals and obtaining the electrical potential is executed entirely using a hearing prosthesis of which the component permanently implanted in the recipient is a part.

21. A device, comprising:
a hearing prosthesis including an implantable component, wherein
the hearing prosthesis is configured to mechanically stimulate tissue of the recipient to evoke a hearing percept, and
the hearing prosthesis is configured to at least one of:
record data based on electrophysiological signals of the recipient resulting from mechanical stimulation of the component(s) by the implantable hearing prosthesis; or
evaluate the data.

22. The device of claim 21, wherein:
the hearing prosthesis includes a direct acoustic cochlear stimulator (DACS) sub-system and an electrophysiology measurement sub-system, wherein
at least a portion of the DACS sub-system and at least a portion of the electrophysiology measurement sub-system are configured to be permanently implanted in a recipient.

23. The device of claim 21, wherein:
the hearing prosthesis includes a bone conduction sub-system and an electrophysiology measurement sub-system, wherein
at least a portion of the bone conduction sub-system and at least a portion of the electrophysiology measurement sub-system are configured to be permanently implanted in a recipient, and the electrophysiology measurement sub-system is configured to record the data based on electrophysiological signals of the recipient.

24. The device of claim 21, wherein:
the implantable component is configured to sense electrical phenomena of the recipient indicative of the electrophysiological signals of the recipient resulting from the mechanical stimulation.

25. The device of claim 21, wherein:
the device is configured to evaluate the data; and
the device is configured to automatically adjust a feature of the implantable component based on the evaluated data.

26. The device of claim 21, wherein:
the implantable component is configured to directly stimulate at least one of a middle ear component or an inner ear component of the recipient.

27. The device of claim 21, wherein:
the device is configured to enable the transmission of the data to a non-hearing prosthesis component.

28. The device of claim 21, wherein:
the implantable component is configured to, with respect an inner ear component, only indirectly stimulate the inner ear component of the recipient.

29. The device of claim 21, wherein:
the hearing prosthesis is configured to record the data based on electrophysiological signals of the recipient resulting from mechanical stimulation of the component(s) by the implantable hearing prosthesis.

30. The device of claim 21, wherein:
the hearing prosthesis is configured to electrically stimulate tissue of the recipient to evoke a hearing percept.
